(19) United States
(12) Patent Application Publication
(10) Pub. No.: US 2026/0138952 A1
(43) Pub. Date: May 21, 2026

(54) HETEROARYLATION OF DIVERSE QUATERNARY CARBON CENTERS OF FREE CARBOXYLIC ACIDS

(71) Applicant: THE SCRIPPS RESEARCH INSTITUTE, La Jolla, CA (US)

(72) Inventors: Jin-Quan YU, San Diego, CA (US); Liang HU, San Diego, CA (US); Guangrong MENG, San Diego, CA (US)

(21) Appl. No.: 19/124,050

(22) PCT Filed: Oct. 13, 2023
(86) PCT No.: PCT/US2023/076843

§ 371 Date: Apr. 24, 2025

Related U.S. Application Data
(60) Provisional application No. 63/420,041, filed on Oct. 27, 2022.

(51) Int. Cl. C07D 213/55; C07D 213/61; C07D 213/64; C07D 213/65; C07D 241/12; C07D 307/54; C07D 333/24

(52) U.S. Cl. CPC C07D 213/55; C07D 213/61; C07D 213/64; C07D 213/65; C07D 241/12; C07D 307/54; C07D 333/24

(57) ABSTRACT

The application provides methods of β- and γ-C(sp³)-H heteroarylation of diverse quaternary carbon centers of free aliphatic carboxylic acids using bidentate pyridine-pyridone ligands. Herein disclosed are methods of Pd(II)-catalyzed mono-selective β- and γ-C(sp³)-H aza-heteroarylation of free carboxylic acids, including a-hydrogen containing substrates, with high yields and high mono-selectivity.

### CROSS REFERENCE TO RELATED APPLICATION

This application claims priority to U.S. provisional patent application No. 63/420,041, which was filed on Oct. 27, 2022, and which is hereby incorporated by reference in its entirety.

### GOVERNMENT SUPPORT

This invention was made with government support under GM084019 awarded by the National Institutes of Health. The government has certain rights in the invention.

### FIELD OF THE INVENTION

The application provides methods of β- and γ-C(sp³)-H heteroarylation of diverse quaternary carbon centers of free aliphatic carboxylic acids using bidentate pyridine-pyridone ligands. Herein disclosed are methods of Pd(II)-catalyzed mono-selective β- and γ-C(sp³)-H aza-heteroarylation of free carboxylic acids, including a-hydrogen containing substrates, with high yields and high mono-selectivity.

### BACKGROUND OF THE INVENTION

Aliphatic acids bearing β-aza-heteroaryls are a promising class of compounds for potential drug molecules (Scheme 1A).^{[1]} Within this context, β-C—H heteroarylation of free carboxylic acids represents a highly valuable approach that could merge diverse heteroaryls and aliphatic acids in medicinal chemistry. In particular, that the sequential heteroarylation and other transformations^{[2,3]} of three methyl C—H bonds in pivalic acid could lead to a unified synthetic platform for the construction of medicinally relevant heteroaryl containing quaternary carbon centers, is envisioned (Scheme 1B).

Although, the use of bidentate directing groups has enabled the use of heteroaryl iodides in C—H arylation,^{[4-6]} the incompatibility with aza-heterocycles when using a weakly coordinating free carboxylic acid substrate remains a significant hurdle for establishing this high-throughput platform.

### Sheme 1A-B. A Sequential C—H Activation Platform for Constructing Quaternary Carbon Centers

### A. Aliphatic Acids as Potential Synthons for β-Aza-Heterocycle-Containing Drug Candidates

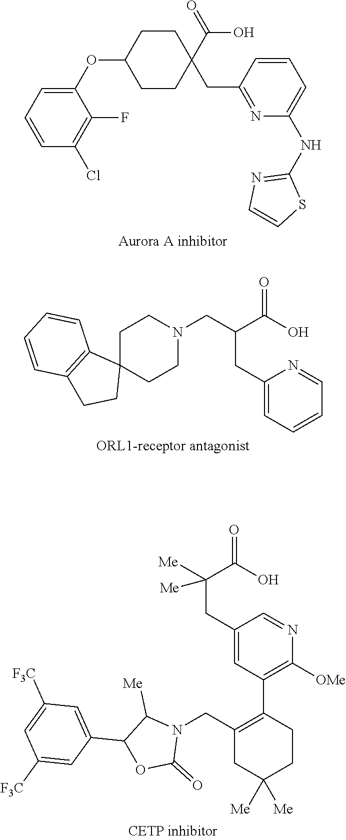 [SMILES: O=C(O)C1(Cc2cccc(Nc3nccs3)n2)CCC(Oc2cccc(Cl)c2F)CC1 ; O=C(O)C(Cc1ccccn1)CN1CCC2(CCc3ccccc32)CC1 ; COc1ncc(CC(C)(C)C(=O)O)cc1C1=C(CN2C(=O)OC(c3cc(C(F)(F)F)cc(C(F)(F)F)c3)C2C)CC(C)(C)CC1]

### B. Modular Synthetic Platform for Diverse Quaternary Carbon Centers

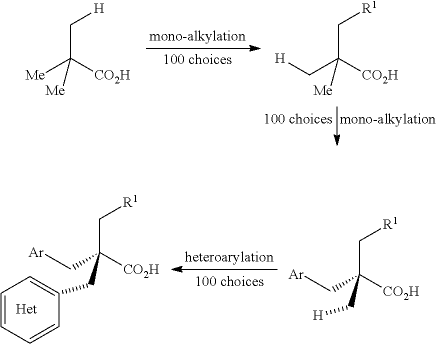

The central challenge is to overcome the competitive coordination of the Lewis basic nitrogen atom with the Pd catalyst that could lead to the formation of thermodynamically favored but catalytically inactive palladium-heteroaryl complex (Scheme 2A).^{[7,8]}

### Sheme 2. β-C(sp³)-H Heteroarylation of Carboxylic Acids

### A. Challenges in Pd-Catalyzed β-Heteroarylation of Free Carboxylic Acids

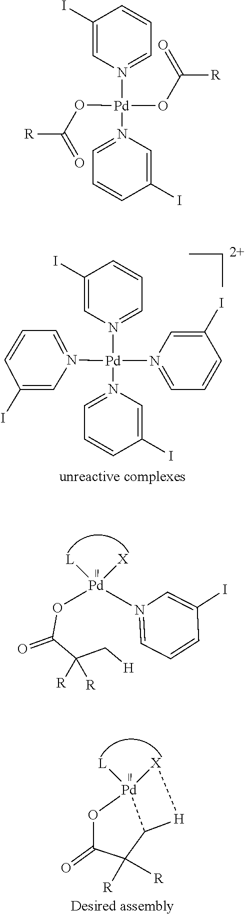

### B. Ligand Enabled β-C(sp³)-H Aza-Heteroarylation of Free Carboxylic Acids (Disclosed Herein)

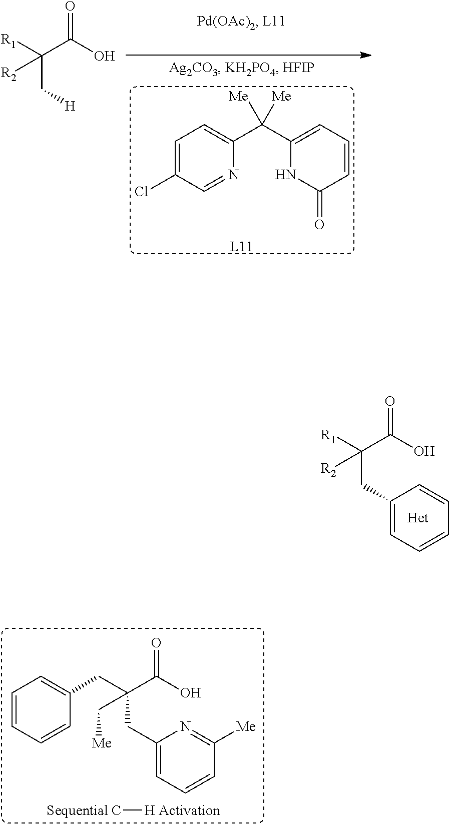

While the use of either strongly coordinating bidentate directing groups^{[4-6]} or 2-substituted iodoheterocycles as the coupling partners^{[9]} can alleviate these adverse effects, the ideal strategy would be to design a suitable ligand capable of promoting the formation of reactive Pd(II) complexes with aliphatic acid substrates in the presence of aza-heterocycles (Scheme 2A), thereby enabling the desired C—H activation step and functionalization steps. In the past decade, a series of ligands to enable/accelerate methyl C—H activation of free aliphatic acid, such as MPAA ligand^{[2f,2j,3a,3b]} and MPAThio ligand^{[2e,2i]} were developed.

Recently, bidentate pyridine-pyridone ligands have been established as a powerful class of ligands for activating previously unreactive C—H bonds.^{[11,12]} For example, the development of a C(sp²)-H hydroxylation reaction with molecular oxygen^{[12]} and β- and γ-methylene C—H dehydrogenation and lactonization reactions directed by native carboxylic acids^{[11]} has been reported.

Yet, there remains a need in the field for effective methods for mono-selective β- and γ-C(sp³)-H aza-heteroarylation of free carboxylic acids that are compatible with high-throughput synthetic requirements. Consequently, since it has recently been found that pyridine-pyridone ligands can in fact tolerate aza-containing heterocyclic substrates,^{[12]} the instant application discloses the exploration of such bidentate pyridine-pyridone ligands as a solution to address the current incompatibility of heteroaryl iodides in C—H activation of free aliphatic acids.

### SUMMARY OF THE INVENTION

Herein disclosed are the first examples of Pd(II)-catalyzed mono-selective β- and γ-C(sp³)-H aza-heteroarylation of free carboxylic acids using bidentate pyridine-pyridone ligands capable of overcoming the current limitation of compatibility with heteroatoms in Pd(II)-catalyzed C(sp³)-H activation of free aliphatic acids (Scheme 2B). This transformation successfully aza-heteroarylates a wide range of free aliphatic carboxylic acids, including the traditionally challenging α-hydrogen containing substrates, with high yields and high mono-selectivity using various non-activated heterocyclic iodides as coupling partners. The sequence of three consecutive mono-selective C(sp³)-H activation reactions of pivalic acid described herein provides a unique platform for constructing diverse quaternary carbon centers containing heteroaryls which has the potential to greatly enhance current capabilities synthetic medicinal chemistry.

The application provides methods of β- or γ-C(sp³)-H heteroarylation of aliphatic carboxylic acids, comprising treating an aliphatic carboxylic acid with a bidentate pyridine-pyridone ligand in the presence of a Pd(II) catalyst.

The application provides the above methods, wherein the method of β- or γ-C(sp³)-H heteroarylation of aliphatic carboxylic acids occurs according to the following reaction scheme:

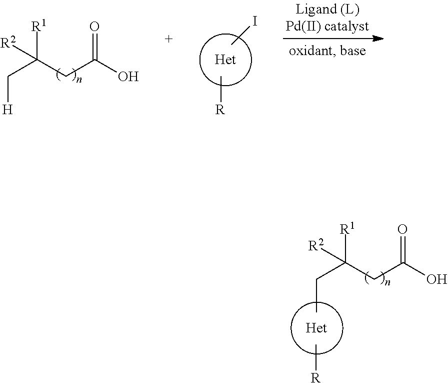

wherein:
Het is (C₅-C₆)heteroaryl;
R is H, halo, OH, (C₁-C₆)alkyl, halo (C₁-C₆)alkyl, halo (C₁-C₆)heteroalkyl, Ac, or —C(═O)H;
R¹ and R² are independently H or selected from the group consisting of (C₁-C₆)alkyl, (C₁-C₆)heteroalkyl, (C₃-C₇)cycloalkyl, (C₃-C₇)heterocycloalkyl, (C₆-C₁₀)aryl, (C₅-C₆)heteroaryl, (C₁-C₆)alkyl (C₃-C₇)cycloalkyl, (C₁-C₆)alkyl (C₃-C₇)heterocycloalkyl, (C₁-C₆)alkyl (C₆-C₁₀)aryl, (C₁-C₆)alkyl (C₅-C₆)heteroaryl, halo (C₁-C₆)alkyl, halo (C₁-C₆)heteroalkyl, halo (C₃-C₇)cycloalkyl, halo (C₃-C₇)heterocycloalkyl, halo (C₆-C₁₀)aryl, halo (C₅-C₆)heteroaryl, halo (C₁-C₆)alkyl (C₃-C₇)cycloalkyl, halo (C₁-C₆)alkyl (C₃-C₇)heterocycloalkyl, halo (C₁-C₆)alkyl (C₆-C₁₀)aryl, and halo (C₁-C₆)alkyl (C₅-C₆)heteroaryl, each independently and optionally substituted with one or more R′;
   each R′ is independently OH, halo, CN, (C₁-C₆)alkyl, halo (C₁-C₆)alkyl, (C₁-C₆)heteroalkyl, (C₃-C₇)cycloalkyl, (C₃-C₇)heterocycloalkyl, NH₂, or —S(═O)₂Me; and n is 0 or 1.

The application further provides a method of β-C(sp³)-H heteroarylation of aliphatic carboxylic acids according to the following reaction scheme:

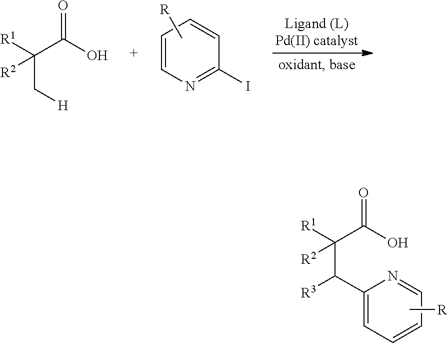

wherein:
R³ is H;
   or R² and R³ together form (C₃-C₄)cycloalkyl.

The application further provides the above method of β-C(sp³)-H heteroarylation of aliphatic carboxylic acids according to the following reaction scheme:

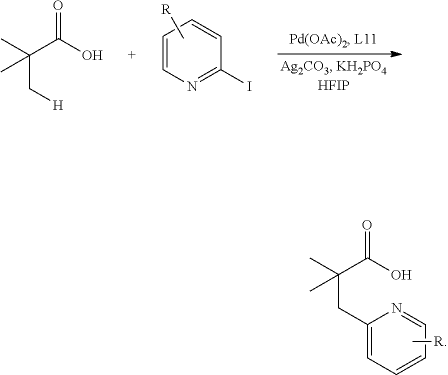

The application further provides a method of γ-C(sp³)-H heteroarylation of aliphatic carboxylic acids according to the following reaction scheme:

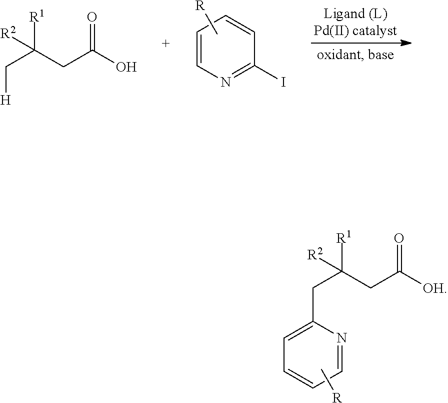

The application further provides the above method of γ-C(sp³)-H heteroarylation of aliphatic carboxylic acids according to the following reaction scheme:

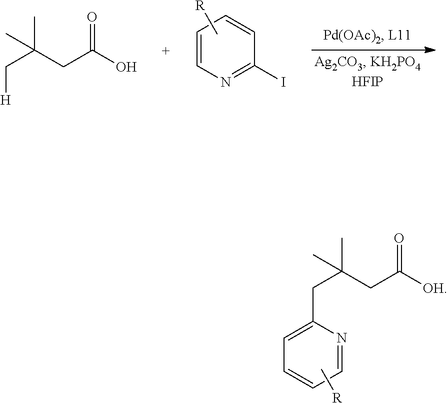

### DETAILED DESCRIPTION OF THE INVENTION

Examples of Pd-catalyzed β-C(sp³)-H aza-heteroarylation of carboxylic acids using a fluorinated aryl amide directing group, albeit limited to 2-substituted iodopyridines have previously been disclosed.^{[9]} In previously reported C(sp³)-H functionalization reactions, it was found that the MPAA or pyridine-based ligands are particularly effective at enabling a wide range of arylation reactions of free carboxylic acids.^{[2b,2c,2g,2h]} However, these protocols suffered from incompatibility with aza-heterocycle aryl iodides and the formation of mono/di arylation mixtures in the presence of two α-methyl groups.

Bearing this in mind, various ligands using pivalic acid 1a and 2-iodopyridine 2a as the model substrates (Table 1) were tested. In the absence of ligand, no product was observed. The monodentate pyridine-type ligand (L1) that was previously utilized to accelerate β-C(sp³)-H olefination of free carboxylic acid failed to give any desired product.^{[2i]} Monodentate pyridone ligand (L2), which was demonstrated to be an efficient ligand to facilitate γ-C(sp³)-H

TABLE 1
Evaluation of Ligands for β-C(sp³)-H Heterarylation.^{[a,b]}

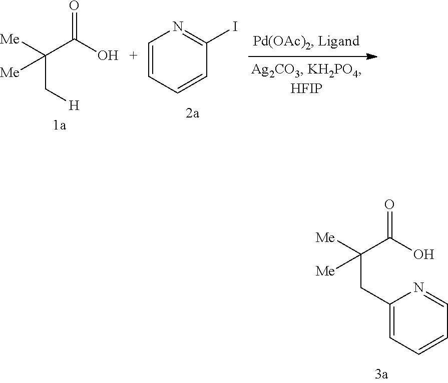 [SMILES: CC(C)(C)C(=O)O ; Ic1ccccn1 ; CC(C)(Cc1ccccn1)C(=O)O]

No ligand
0% 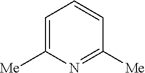
L1, 0% yield 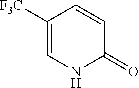 [SMILES: O=c1ccc(C(F)(F)F)c[nH]1]
L2, 0% yield 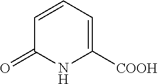
L3, 0% yield 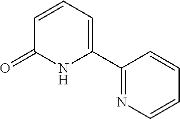 [SMILES: O=c1cccc(-c2ccccn2)[nH]1]
L4, 0% yield 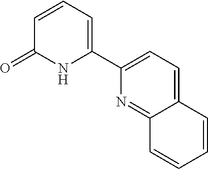 [SMILES: O=c1cccc(-c2ccc3ccccc3n2)[nH]1]
L5, <5% yield 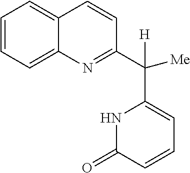 [SMILES: CC(c1ccc2ccccc2n1)c1cccc(=O)[nH]1]
L6, <5% yield 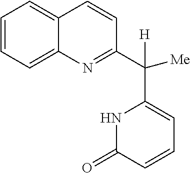 [SMILES: CC(c1ccc2ccccc2n1)c1cccc(=O)[nH]1]
L7, 17% yield 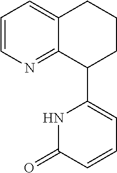 [SMILES: O=c1cccc(C2CCCc3cccnc32)[nH]1]
L8, 0% yield 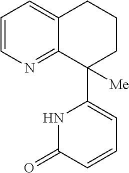 [SMILES: CC1(c2cccc(=O)[nH]2)CCCc2cccnc21]
L9, 39% yield 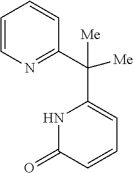 [SMILES: CC(C)(c1ccccn1)c1cccc(=O)[nH]1]
L10, 55% yield 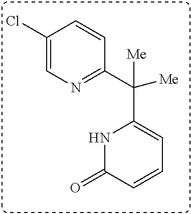 [SMILES: CC(C)(c1ccc(Cl)cn1)c1cccc(=O)[nH]1]
L11, 71% yield 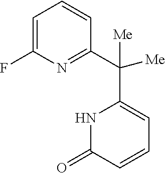 [SMILES: CC(C)(c1cccc(F)n1)c1cccc(=O)[nH]1]
L12, 21% yield 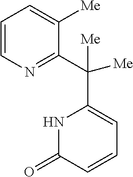 [SMILES: Cc1cccnc1C(C)(C)c1cccc(=O)[nH]1]
L13, 60% yield 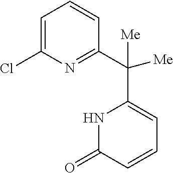 [SMILES: CC(C)(c1cccc(Cl)n1)c1cccc(=O)[nH]1]
L14, 40% yield 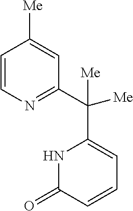 [SMILES: Cc1ccnc(C(C)(C)c2cccc(=O)[nH]2)c1]
L15, 37% yield 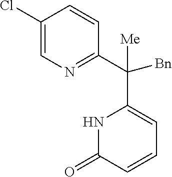 [SMILES: CC(Cc1ccccc1)(c1ccc(Cl)cn1)c1cccc(=O)[nH]1]
L16, 53% yield
^{[a]}Conditions: 1a (0.1 mmol), 2a (2.0 equiv.), Pd(OAc)₂ (10 mol %), ligand (10 mol %), KH₂PO₄ (3.0 equiv.), Ag₂CO₃ (2.0 equiv.), HFIP (1.0 M), 120° C., under air, 24 h.
^{[b]1}H NMR yields, using CH₂Br₂ as an internal standard.

heteroarylation of ketones directed by pre-installed bidentate directing groups also did not provide any product.^{5f} The pyridone-based bidentate ligands were next investigated. Recent findings showed that these types of bidentate ligands can stabilize the palladium catalyst and promote methylene C—H cleavage.^{[11]}

Importantly, previous efforts toward C—H hydroxylation showed that pyridone-pyridine bidentate ligands can tolerate the aza-arylbenzoic acid substrates.^{[12]} Excitingly, heteroarylated product was first observed with this type of bidentate ligand (L5) that forms a five-membered palladacycle, albeit in low yield (<5% yield by 1H NMR analysis). This result then encouraged aiming to accelerate the C—H bond

TABLE S1
Ligand effects for β-C(sp³)-H heteroarylation^{a,b}

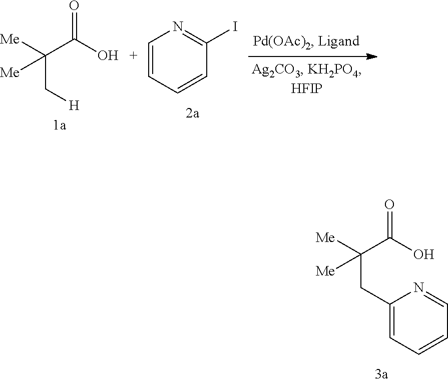 [SMILES: CC(C)(C)C(=O)O ; Ic1ccccn1 ; CC(C)(Cc1ccccn1)C(=O)O]

No ligand
0% 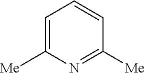
L1, 0% yield 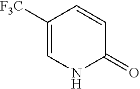 [SMILES: O=c1ccc(C(F)(F)F)c[nH]1]
L2, 0% yield 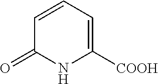
L3, 0% yield 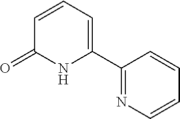 [SMILES: O=c1cccc(-c2ccccn2)[nH]1]
L4, 0% yield 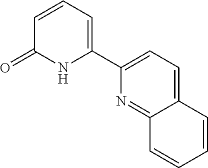 [SMILES: O=c1cccc(-c2ccc3ccccc3n2)[nH]1]
L5, <5% yield 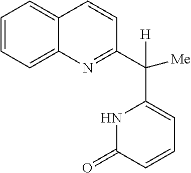 [SMILES: CC(c1ccc2ccccc2n1)c1cccc(=O)[nH]1]
L6, <5% yield 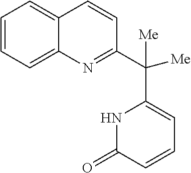 [SMILES: CC(C)(c1ccc2ccccc2n1)c1cccc(=O)[nH]1]
L7, 17% yield 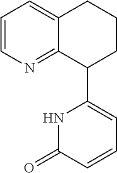 [SMILES: O=c1cccc(C2CCCc3cccnc32)[nH]1]
L8, 0% yield 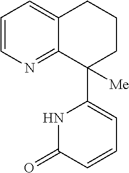 [SMILES: CC1(c2cccc(=O)[nH]2)CCCc2cccnc21]
L9, 39% yield 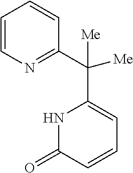 [SMILES: CC(C)(c1ccccn1)c1cccc(=O)[nH]1]
L10, 55% yield 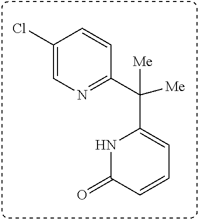 [SMILES: CC(C)(c1ccc(Cl)cn1)c1cccc(=O)[nH]1]
L11, 71% yield 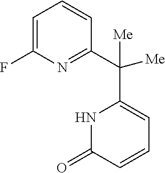 [SMILES: CC(C)(c1cccc(F)n1)c1cccc(=O)[nH]1]
L12, 21% yield 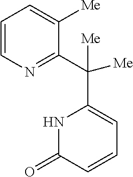 [SMILES: Cc1cccnc1C(C)(C)c1cccc(=O)[nH]1]
L13, 60% yield 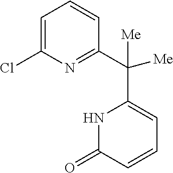 [SMILES: CC(C)(c1cccc(Cl)n1)c1cccc(=O)[nH]1]
L14, 40% yield 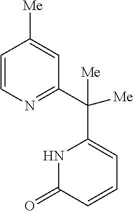 [SMILES: Cc1ccnc(C(C)(C)c2cccc(=O)[nH]2)c1]
L15, 37% yield 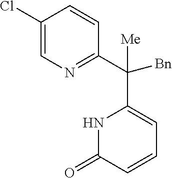 [SMILES: CC(Cc1ccccc1)(c1ccc(Cl)cn1)c1cccc(=O)[nH]1]
L16, 53% yield 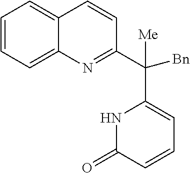 [SMILES: CC(Cc1ccccc1)(c1ccc2ccccc2n1)c1cccc(=O)[nH]1]
L17, 22% yield 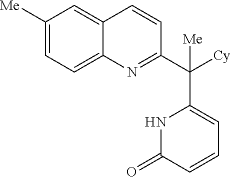
L18, 8% yield 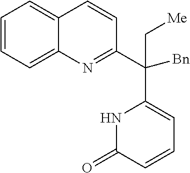 [SMILES: CCC(Cc1ccccc1)(c1ccc2ccccc2n1)c1cccc(=O)[nH]1]
L19, 16% yield 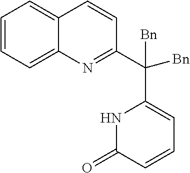 [SMILES: O=c1cccc(C(Cc2ccccc2)(Cc2ccccc2)c2ccc3ccccc3n2)[nH]1]
L20, 11% yield 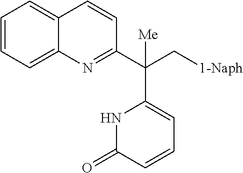
L21, 13% yield 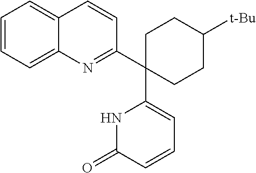 [SMILES: CC(C)(C)C1CCC(c2ccc3ccccc3n2)(c2cccc(=O)[nH]2)CC1]
L22, 17% yield 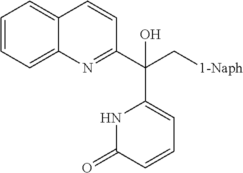
L23, <5% yield 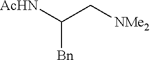 [SMILES: CC(=O)NC(Cc1ccccc1)CN(C)C]
L24, 9% yield 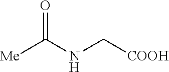
L25, <5% yield
^{a}Conditions: 1a (0.1 mmol), 2a (2.0 equiv.), Pd(OAc)₂ (10 mol %), ligand (10 mol %), KH₂PO₄ (3.0 equiv.), Ag₂CO₃ (2.0 equiv.), HFIP (0.1 mL), 120° C., under air, 24 h.
^{b}1H NMR yields, using CH₂Br₂ as an internal standard.

cleavage through changing the ligand bite angle (L6 and L7). Indeed, the ligand L7 with a six-membered chelation improved the yield to 17%. Efforts to modify the substitution on the ligand backbone did not further enhance the reactivity (Table S1). However, the switch from quinoline to 5,6,7,8-tetrahydroquinoline dramatically improved the yield to 39% (L8 and L9). These results showed that the catalytic performance of these catalysts critically depends on the structure of the ligands. As expected, the yield was further improved to 55% with pyridine-pyridone ligand L10. Through systematic variation of the substituents at different positions on the pyridine ring and backbone of ligand (L11-L16), 5-chloropyridine-pyridone ligand (L11) was eventually identified to afford 71% yield with high mono-selectivity.

Aside from the ligand, the reaction concentration was found to be critical for achieving high yields of the desired products (Table S2). In order to obtain the above stated 71% with L11, the concentration of the reaction was increased to 1.0 M. Using a 0.1 M concentration, which is typically used in such reaction development, in combination with L11 did not provide any of the desired heteroarylated product. In the absence of any solvent, 27% yield of the product was obtained. It is surmised that high concentration is beneficial for the interactions of the aryl-iodide bond with the Pd(II) center, which is essential for the arylation to proceed.

TABLE S2
Concentration effects for β-C(sp³)-H heteroarylation^{a,b}

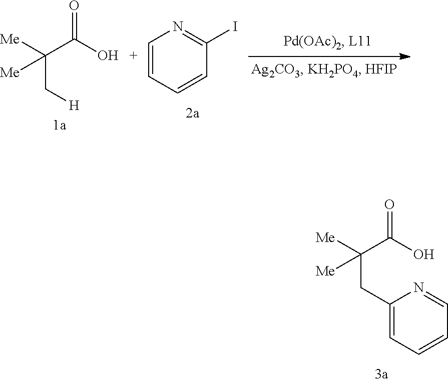 [SMILES: CC(C)(C)C(=O)O ; Ic1ccccn1 ; CC(C)(Cc1ccccn1)C(=O)O]

Concentration effect:

| Conc. (M) | Yield |
|---|---|
| 0.1 | 0% |
| 0.2 | <5% |
| 0.5 | 35% |
| 1.0 | 71% |
| 2.0 | 44% |
| neat | 27% |

^{a}Conditions: 1a (0.1 mmol), 2a (2.0 equiv.), Pd(OAc)₂ (10 mol %), L11 (10 mol %), KH₂PO₄ (3.0 equiv.), Ag₂CO₃ (2.0 equiv.), HFIP (x mol/L), 120° C., under air, 24 h.
^{b}1H NMR yields, using CH₂Br₂ as an internal standard.

With the optimal conditions in hand, various heterocyclic iodides as coupling partners with pivalic acid (Table 2) were examined. Heteroarylation with various strongly coordinating unsubstituted 2-iodopyridines proceeded smoothly with high mono-selectivity regardless of their electronic properties (3a-3f). Not surprisingly, the more reactive 2-substituted pyridines were all compatible (3g-3j) and provided the desired products in good to excellent yields.

When the sterically demanding 2-iodopyridine bearing an ortho methoxy substituent was used in the reaction, the corresponding product 3k in 35% yield was observed. Moderate product yields were obtained with the unsubstituted 3-iodopyridines (3l, 3m). The use of activated 3-iodopyridines (3n-3q) and 4-iodopyridines (3r-3u) bearing a variety of functionalities resulted in products being formed in overall good yields.

However, possibly due to the high reactivity of heterocyclic coupling partners 3n, 3q, and 3r, small amounts of di-heteroarylated products were observed in these three cases. It is worth noting that heteroaryl iodides containing pyridines with different substitutions such as 2-fluoro-(3n), chloro-(3c, 3o, 3s), and bromo-(3d, 3h, 3m, 3p and 3t) groups at different positions were all compatible as coupling partners generating products with useful synthetic handles, which can be used for subsequent product derivatization.

Additional heterocycle iodides such as 2-iodopyrazine (3v), iodo-thiophene (3w), and iodo-furan (3x) were also suitable coupling partners which further demonstrates the generality of this reaction protocol.

TABLE 2
Scope of the Heterocyclic Iodides for β-C(sp³)-H Heterarylation.^{[a,b,c,d]}

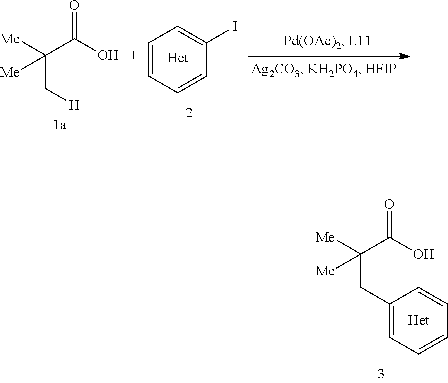 [SMILES: CC(C)(C)C(=O)O ; Ic1ccccc1 ; CC(C)(Cc1ccccc1)C(=O)O]

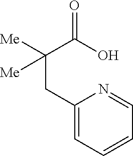 [SMILES: CC(C)(Cc1ccccn1)C(=O)O]

3a, 70% yield 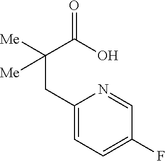 [SMILES: CC(C)(Cc1ccc(F)cn1)C(=O)O]
3b, 59% yield 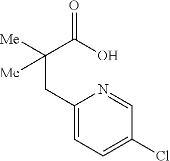 [SMILES: CC(C)(Cc1ccc(Cl)cn1)C(=O)O]
3c, 74% yield 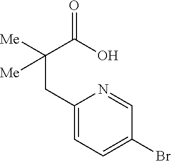 [SMILES: CC(C)(Cc1ccc(Br)cn1)C(=O)O]
3d, 67% yield 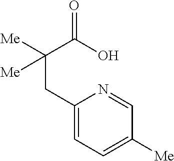 [SMILES: Cc1ccc(CC(C)(C)C(=O)O)nc1]
3e, 62% yield 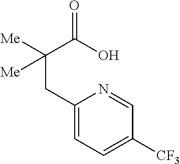 [SMILES: CC(C)(Cc1ccc(C(F)(F)F)cn1)C(=O)O]
3f, 41% yield 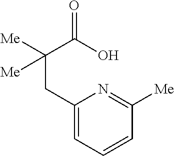 [SMILES: Cc1cccc(CC(C)(C)C(=O)O)n1]
3g, 72% yield 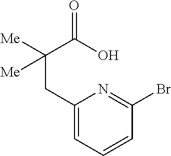 [SMILES: CC(C)(Cc1cccc(Br)n1)C(=O)O]
3h, 55% yield 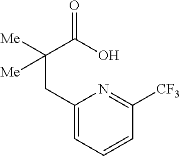 [SMILES: CC(C)(Cc1cccc(C(F)(F)F)n1)C(=O)O]
3i, 66% yield 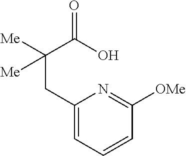 [SMILES: COc1cccc(CC(C)(C)C(=O)O)n1]
3j, 75% yield 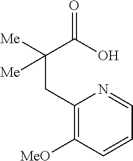 [SMILES: COc1cccnc1CC(C)(C)C(=O)O]
3k, 35% yield^{c} 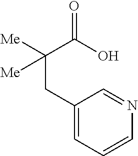 [SMILES: CC(C)(Cc1cccnc1)C(=O)O]
3l, 46% yield^{c,d} 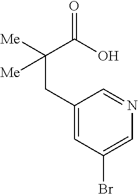 [SMILES: CC(C)(Cc1cncc(Br)c1)C(=O)O]
3m, 33% yield^{c,d} 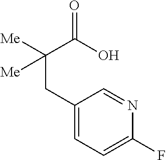 [SMILES: CC(C)(Cc1ccc(F)nc1)C(=O)O]
3n, 60% yield^{c} mono:di = 4:1 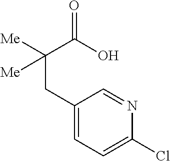 [SMILES: CC(C)(Cc1ccc(Cl)nc1)C(=O)O]
3o, 62% yield 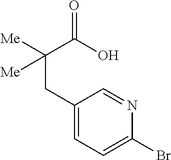 [SMILES: CC(C)(Cc1ccc(Br)nc1)C(=O)O]
3p, 53% yield 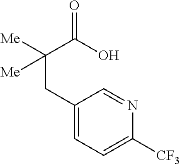 [SMILES: CC(C)(Cc1ccc(C(F)(F)F)nc1)C(=O)O]
3q, 70% yield mono:di = 10:1 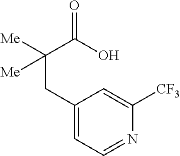 [SMILES: CC(C)(Cc1ccnc(C(F)(F)F)c1)C(=O)O]
3r, 68% yield mono:di = 9:1 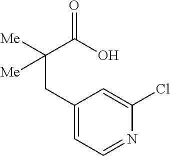 [SMILES: CC(C)(Cc1ccnc(Cl)c1)C(=O)O]
3s, 56% yield 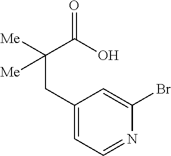 [SMILES: CC(C)(Cc1ccnc(Br)c1)C(=O)O]
3t, 43% yield 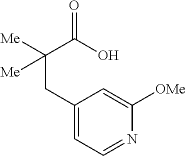 [SMILES: COc1cc(CC(C)(C)C(=O)O)ccn1]
3u, 50% yield 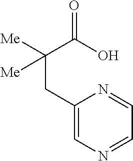 [SMILES: CC(C)(Cc1cnccn1)C(=O)O]
3v, 54% yield 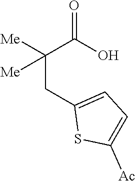 [SMILES: CC(=O)c1ccc(CC(C)(C)C(=O)O)s1]
3w, 44% yield 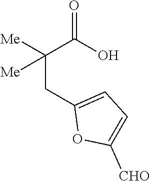 [SMILES: CC(C)(Cc1ccc(C=O)o1)C(=O)O]
3x, 39% yield^{d}
^{[a]}Conditions: 1a (0.1 mmol), 2 (2.0 equiv.), Pd(OAc)₂ (10 mol %), L11 (10 mol %), KH₂PO₄ (3.0 equiv.), Ag₂CO₃ (2.0 equiv.), HFIP (1.0 M), 120° C., under air, 24 h.
^{[b]}Isolated yield.
^{[c]}48 h.
^{[d]}HFIP (0.2 ml).

Next, the scope of this C(sp³)-H heteroarylation with respect to the carboxylic acids was surveyed. The protocol was applicable to a variety of substrates (Table 3). A wide range of quaternary aliphatic acids (4a-4l) bearing α-gem-dimethyl groups with various aliphatic chains were all compatible, affording the β-heteroarylated products in 60-86% yields. Among these

TABLE 3
Carboxylic Acid Scope for β-C(sp³)-H Heteroarylation.^{[a,b]}

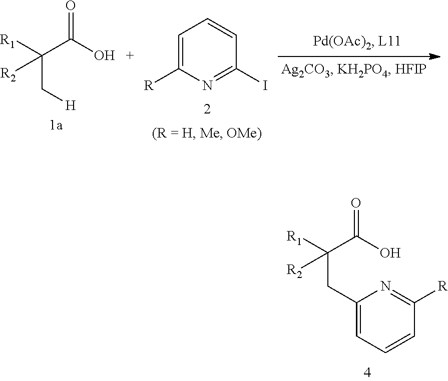

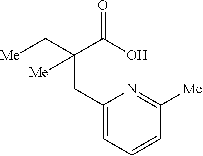 [SMILES: CCC(C)(Cc1cccc(C)n1)C(=O)O]

4a, 77% yield 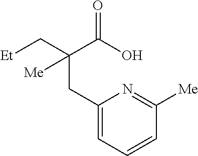 [SMILES: CCCC(C)(Cc1cccc(C)n1)C(=O)O]
4b, 86% yield 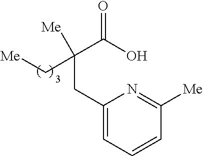 [SMILES: CCC(C)(Cc1cccc(C)n1)C(=O)O]
4c, 73% yield 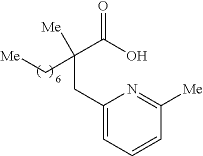 [SMILES: CCC(C)(Cc1cccc(C)n1)C(=O)O]
4d, 65% yield 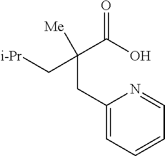 [SMILES: CC(C)CC(C)(Cc1ccccn1)C(=O)O]
4e, 60% yield 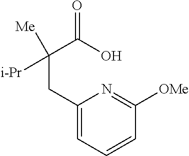 [SMILES: COc1cccc(CC(C)(C(=O)O)C(C)C)n1]
4f, 62% yield 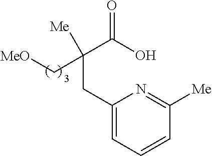 [SMILES: COCC(C)(Cc1cccc(C)n1)C(=O)O]
4g, 62% yield 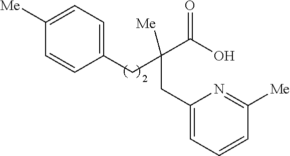 [SMILES: Cc1ccc(CC(C)(Cc2cccc(C)n2)C(=O)O)cc1]
4h, 81% yield 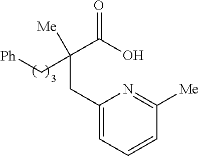 [SMILES: Cc1cccc(CC(C)(Cc2ccccc2)C(=O)O)n1]
4i, 70% yield 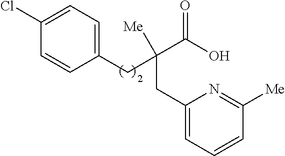 [SMILES: Cc1cccc(CC(C)(Cc2ccc(Cl)cc2)C(=O)O)n1]
4j, 67% yield 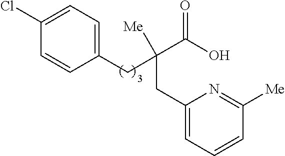 [SMILES: Cc1cccc(CC(C)(Cc2ccc(Cl)cc2)C(=O)O)n1]
4k, 62% yield 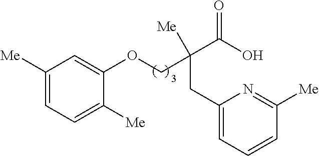 [SMILES: Cc1ccc(C)c(OCC(C)(Cc2cccc(C)n2)C(=O)O)c1]
4l, 62% yield 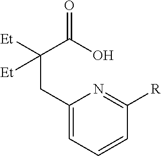
4m, R = H, 40% yield 4n, R = Me, 41% yield 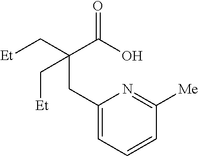 [SMILES: CCCC(CCC)(Cc1cccc(C)n1)C(=O)O]
4o, 46% yield 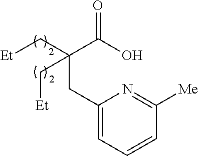
4p, 55% yield 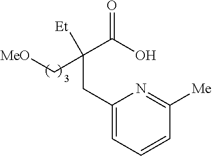 [SMILES: CCC(COC)(Cc1cccc(C)n1)C(=O)O]
4q, 41% yield 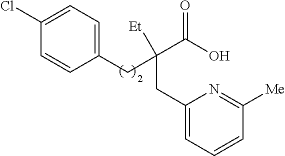 [SMILES: CCC(Cc1ccc(Cl)cc1)(Cc1cccc(C)n1)C(=O)O]
4r, 40% yield 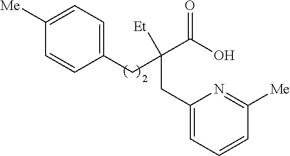 [SMILES: CCC(Cc1ccc(C)cc1)(Cc1cccc(C)n1)C(=O)O]
4s, 43% yield 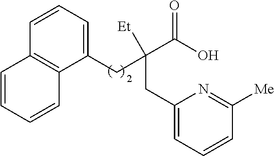 [SMILES: CCC(Cc1cccc(C)n1)(Cc1cccc2ccccc12)C(=O)O]
4t, 50% yield 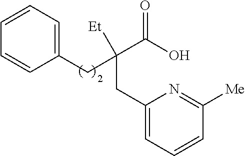 [SMILES: CCC(Cc1ccccc1)(Cc1cccc(C)n1)C(=O)O]
4u, 44% yield 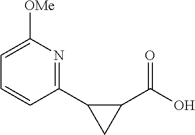 [SMILES: COc1cccc(C2CC2C(=O)O)n1]
4v, 45% yield 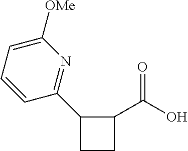 [SMILES: COc1cccc(C2CCC2C(=O)O)n1]
4w, 41% yield 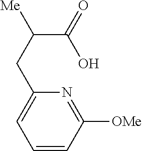 [SMILES: COc1cccc(CC(C)C(=O)O)n1]
4x, 55% yield 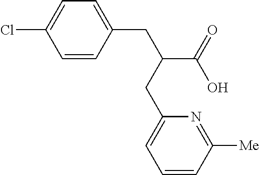 [SMILES: Cc1cccc(CC(Cc2ccc(Cl)cc2)C(=O)O)n1]
4y, 51% yield
^{[a]}Conditions: 1 (0.1 mmol), 2 (2.0 equiv.), Pd(OAc)₂ (10 mol %), L11 (10 mol %), KH₂PO₄ (3.0 equiv.), Ag₂CO₃ (2.0 equiv.), HFIP (1.0 M), 120° C., under air, 24 h.
^{[b]}Isolated yield.

results, Gemfibrozil (4l), an oral drug used to lower lipid levels,^{[13]} could be heteroarylated in 62% yield. Moderate yields could be observed with a variety of acids containing a single α-methyl group (4m-4u). The presence of halogens in the heteroarylated products 4j, 4k and 4r offer a synthetic handle for subsequent derivatization. Given the highly mono-selective nature of this protocol, the remaining α-methyl substituent from the products 4a-4l could subsequently undergo additional C—H functionalization reactions to afford derivatized products with large structural diversities. This strategy was also successfully extended to the heteroarylation of α-hydrogen containing cyclopropyl 4v and cyclobutyl 4w C—H bonds with moderate yields. Lastly, the α-hydrogen containing carboxylic acids 4x and 4y were both compatible under the developed reaction conditions and afforded the corresponding products in 55% and 51% yields. It is worth noting that the α-hydrogen substrates lacking the favorable Thorpe-Ingold effect as well as the interfering acidic α-C—H bond are generally challenging substrates for C—H functionalization reactions.

In order to gain insight into the role of ligand and the origin for high mono-selectivity, preliminary mechanistic studies were conducted under the standard condition. Taken together, it can reasonably be concluded from these results that: (1) the bidentate pyridine-pyridone ligand is crucial for C—H activation by overcoming the non-productive coordination of the nitrogen atoms of the heteroaryl iodides; and (2) the monoarylated product can still undergo C—H activation, but not the subsequent oxidative addition or reductive elimination with heteroaryl iodides.

Since the protocol worked well with β-C(sp³)-H bonds, it was questioned whether this methodology could be extended to functionalize distal γ-C(sp³)-H bonds of free aliphatic acids. γ-Heteroarylated free acids cannot be accessed through traditional carbonyl reactivity; therefore, such transformation would represent a novel disconnection to synthesize free carboxylic acids possessing a γ-substituted heterocycle. Indeed, γ-C(sp³)-H heteroarylation of 5 with a variety of iodopyridines proceeded smoothly using the standard reaction conditions and provided products 6a-6f in moderate yields (Table 4). Of note, the 6-position unsubstituted 2-iodopyridines 6a and 6b and the substituted 2-iodopyridine 6c displayed similar efficiencies. These results demonstrate the ability of the newly developed ligand to overcome the limitations of conventional methodologies and underscore the transformative power of C—H functionalization reactions.

TABLE 4
γ-C(sp³)-H Heteroarylation of Carboxylic Acids.^{[a,b]}

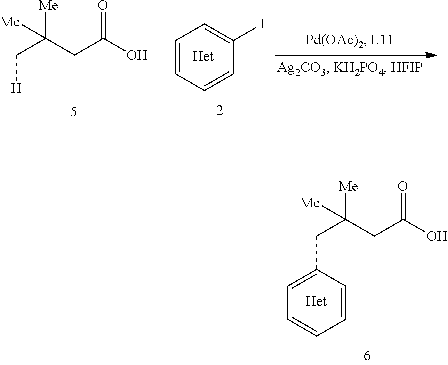

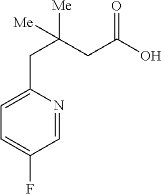 [SMILES: CC(C)(CC(=O)O)Cc1ccc(F)cn1]

6a, 48% yield 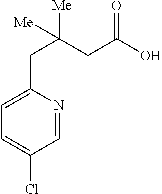 [SMILES: CC(C)(CC(=O)O)Cc1ccc(Cl)cn1]
6b, 44% yield 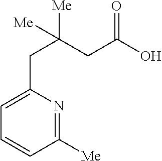 [SMILES: Cc1cccc(CC(C)(C)CC(=O)O)n1]
6c, 56% yield 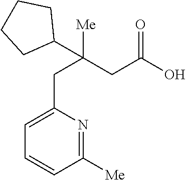 [SMILES: Cc1cccc(CC(C)(CC(=O)O)C2CCCC2)n1]
6d, 44% yield 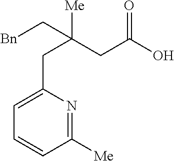 [SMILES: Cc1cccc(CC(C)(CCc2ccccc2)CC(=O)O)n1]
6e, 42% yield 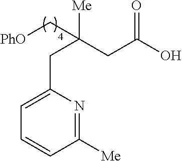
6f, 52% yield
^{[a]}Conditions: 5 (0.1 mmol), 2 (2.0 equiv.), Pd(OAc)₂ (10 mol %), L11 (10 mol %), KH₂PO₄ (3.0 equiv.), Ag₂CO₃ (2.0 equiv.), HFIP (0.2 M), 120° C., under air, 24 h.
^{[b]}Isolated yield.

The high mono-selectivity and compatibility with heteroaryl iodide paved the way to establish a unique synthetic platform for constructing diverse tertiary and quaternary carbon centers containing heteroaryls (Scheme 3). Sequentially activating multiple C—H bonds in one aliphatic acid substrate provides a highly versatile access to diverse molecular architectures. Subsequently, the sequential di-functionalization of isobutyric acid (1y) was embarked upon. Using a MPAA ligand, the mono-arylated product 1z in 70% yield^{[2b]} was obtained, which was further aza-heteroarylated to give 4y in 51% yield. Next, this protocol was extrapolated to obtain a tri-functionalized product from pivalic acid (1a) via a sequence of three consecutive C(sp³)-H activation reactions. Starting with a β-C(sp³)-H lactonization of pivalic acid,^{[3a]} the corresponding β-lactone that was opened with Grignard as the nucleophile to afford 1b in 45% yield was obtained. This product was then arylated with iodobenzene to generate 7 in 72% yield.^{[2a]} Lastly, using the heteroarylation protocol developed herein, the 2-methyl pyridine was installed to the remaining methyl substituent and the trisubstituted aliphatic acid 8 was obtained in 52% yield.

### Scheme 3A-B. Sequential C—H Functionalization

In summary, the first examples of palladium-catalyzed β- and γ-C(sp³)-H aza-heteroarylation of free carboxylic acids enabled by a newly developed pyridine-pyridone ligand are herein described. This methodology is compatible with a wide range of carboxylic acids and features a diverse functional group tolerance. The use of non-activated pyridine iodides as coupling partners renders this reaction highly general and practical. The synthetic utility of this reaction is demonstrated by preparation of a heteroaryl-containing stereocenter from isobutyric acid and pivalic acid via a sequence of two or three consecutive C(sp³)-H activation reactions.

### REFERENCES

[1] a) G. D. Henry, Tetrahedron 2004, 60, 6043-6061; b) P. N. Kalaria, S. C. Karad, D. K. Raval, Eur. J. Med. Chem. 2018, 158, 917-936; c) Modern Heterocyclic Chemistry, 1st ed. (Eds.: J. Elguero, A. M. S. Silva, A. C. Tomé), Wiley-VCH, Weinheim, 2011, 635-725.
[2] For C(sp³)-C bond formation of free acid, see: a) R. Giri, N. Maugel, J.-J. Li, D.-H. Wang, S. P. Breazzano, L. B. Saunder, J.-Q. Yu, J. Am. Chem. Soc. 2007, 129, 3510-3511; b) Y. Zhu, X. Chen, C. Yuan, G. Li, J. Zhang, Y. Zhao, Nat. Commun. 2017, 8, 14904-14911; c) K. K. Ghosh, M. van Gemmeren, Chem.-Eur. J. 2017, 23, 17697-17700; d) F. Ghiringhelli, A. Uttry, K. K. Ghosh, M. van Gemmeren, Angew. Chem., Int. Ed. 2020, 59, 23127-23131; Angew. Chem. 2020, 132, 23327-23331; e) Z. Zhuang, A. N. Herron, J.-Q. Yu, Angew. Chem., Int. Ed. 2021, 60, 16382-16387; Angew. Chem. 2021, 133, 16518-16523; f) H. S. Park, Z. L. Fan, R.-Y. Zhu, J.-Q. Yu, Angew. Chem., Int. Ed. 2020, 59, 12853-12859; Angew. Chem. 2020, 132, 12953-12959 g) P. Dolui, J. Das, H. B. Chandrashekar, S. S. Anjana, D. Maiti, Angew. Chem., Int. Ed. 2019, 58, 13773-13777; Angew. Chem. 2019, 131, 13911-13915; h) G. Chen, Z. Zhuang, G.-C.; Li, T. G. Saint-Denis, Y. Hsiao, C. L. Joe, J.-Q. Yu, Angew. Chem., Int. Ed. 2017, 56, 1506-1509; Angew. Chem. 2017, 129, 1528-1531; i) Z. Zhuang, C.-B. Yu, G. Chen, Q.-F. Wu, Y. Hsiao, C. L. Joe, J. X. Qiao, M. A. Poss, J.-Q. Yu, J. Am. Chem. Soc. 2018, 140, 10363-10367; j) Z, Zhuang, A. N. Herron, S. Liu, J.-Q. Yu, J. Am. Chem. Soc. 2021, 143, 687-692; k) L. Hu, P.-X. Shen, Q. Shao, K. Hong, J.-Q. Yu, Angew. Chem., Int. Ed. 2019, 58, 2134-2138; Angew. Chem. 2019, 131, 2156-2160.
[3] For C(sp³)-X bond formation of acid, see: a) Z. Zhuang, J.-Q. Yu, Nature 2020, 577, 656-659; b) Z. Zhuang, A. N. Herron, Z. L. Fan, J.-Q. Yu, J. Am. Chem. Soc. 2020, 142, 6769-6776; c) K. K. Ghosh, A. Uttry, A. Koldemir, M. Ong, M. van Gemmeren, Org. Lett. 2019, 21, 7154-7157; d) P. Novak, A. Correa, J. Gallardo-Donaire, R. Matrin, Angew. Chem., Int. Ed. 2011, 50, 12236-12239; Angew. Chem. 2011, 123, 12444-12447.
[4] For reviews, see: a) S. Rej, Y. Ano, N. Chatani, Chem. Rev. 2019, 120, 1788-1887; b) J. He, M. Wasa, K. S. L. Chan, Q. Shao, J.-Q. Yu, Chem. Rev. 2017, 117, 8754-8786; c) B. Liu, R. C. Z. Romine, K. M. Engle, B.-F. Shi, Chem. Rev. 2021, 121, 14957-15074; d) O. Daugulis, J. Roane, L. D. Tran, Acc. Chem. Res. 2015, 48, 1053-1064; e) E. L. Lucas, N. Y. S. Lam, Z. Zhuang, H. S. S. Chan, D. A. Strassfeld, J.-Q. Yu, Acc. Chem. Res. 2022, 55, 537-550.
[5] For selected examples using bidentate directing groups, see: a) J. Ghouilem, C. Tran, N. Grimblat, P. Retailleau, M. Alami, V. Gandon, S. Messaoudi, ACS Catal. 2021, 11, 1818-1826; b) N. Hoshiya, T. Kobayashi, M. Arisawa, S. Shuto, Org. Lett. 2013, 15, 6202-6205; c) M. D. Reddy, E. B. Watkins, J. Org. Chem. 2015, 80, 11447-11459; d) D. P. Affron, O. A. Davis, J. A. Bull, Org. Lett. 2014, 16, 4956-4959; e) X. Yang, M. X. Jiang, T. Zhou, Y. Q. Han, X. T. Xu, K. Zhang, B.-F. Shi, Chem. Commun. 2021, 57, 5562-5565; f) R.-Y. Zhu, Z.-Q. Li, H. S. Park, C. H. Senanayake, J.-Q. Yu, J. Am. Chem. Soc. 2018, 140, 3564-3568; g) R.-Y. Zhu, L.-Y. Liu, H. S. Park, K. Hong, Y. W. Wu, C. H. Senanayake, J.-Q. Yu, J. Am. Chem. Soc. 2017, 139, 16080-16083; h) G. Q. Xia, Z. Zhuang, L.-Y. Liu, S. L. Schreiber, B. Melillo, J.-Q. Yu, Angew. Chem., Int. Ed. 2020, 59, 7783-7787; Angew. Chem. 2020, 132, 7857-7861.
[6] For selected examples using bidentate transit-directing groups, see: a) Y.-Q. Chen, Z. Wang, Y. W. Wu, S. R. Wisniewski, J. X. Qiao, W. R. Ewing, M. D. Eastgate, J.-Q. Yu, J. Am. Chem. Soc. 2018, 140, 17884-17894; b) Y. B. Liu, H. B. Ge, Nature Chem. 2017, 9, 26-32.
[7] a) A. K. Cook, M. S. Sanford, J. Am. Chem. Soc. 2015, 137, 3109-3118; b) M. C. Ye, G.-L. Gao, J.-Q. Yu, J. Am. Chem. Soc. 2011, 133, 6964-6967; c) Z. P. Zhang, K. Tanaka, J.-Q. Yu, Nature 2017, 543, 538-542.
[8] a) K. M. Engle, T.-S. Mei, M. Wasa, J.-Q. Yu, Acc. Chem. Res. 2012, 45, 788-802; b) K. M. Engle, J.-Q. Yu, J. Org. Chem. 2013, 78, 8927-8955.
[9] a) J. He, S. H. Li, Y. Q. Deng, H. Y. Fu, B. N. Laforteza, J. E. Spangler, A. Homs, J.-Q. Yu, Science 2014, 343, 1216-1220; b) G. Chen, T. Shigenari, P. Jain, Z. Zhang, Z. Jin, J. He, S. Li, C. Mapelli, M. M. Miller, M. A. Poss, P. M. Scola, K.-P. Yeung, J.-Q. Yu, J. Am. Chem. Soc. 2015, 137, 3338-3351.
[10] Z. Fan, S. Zhao, T. Liu, P.-X. Shen, Z.-N. Cui, Z. Zhuang, Q. Shao, J. S. Chen, A. S. Ratnayake, M. E. Flanagan, D. K. Kölmel, D. W. Piotrowski, P. Richardson, J.-Q. Yu, Chem. Sci. 2020, 11, 12282-12288. In this report, DNA-attached free acid were employed as directing groups for C(sp³)-H aza-heteroarylation with MPAAM ligands.
[11] a) Z. Wang, L. Hu, N. Cheksin, Z. Zhuang, S.-Q. Qian, J.-X. Qiao, J.-Q. Yu, Science 2021, 374, 1281-1285; b) H. S. S. Chan, J.-M. Yang, J.-Q. Yu, Science 2022, 376, 1481-1487; c) T. Sheng, Z. Zhuang, Z. Wang, L. Hu, A. N. Herron, J. X. Qiao, J.-Q. Yu, J. Am. Chem. Soc. 2022, 144, 12924-12933.
[12] Z. Li, Z. Wang, N. Cheksin, S. Q. Qian, J. X. Qiao, P. Cheng, K.-S. Yeung, W. R. Ewing, J.-Q. Yu, Science 2021, 372, 1452-1457.
[13] C. Pueyo Palazón, J. Alfón, P. Gaffney, M. Berrozpe, T. Royo, L. Badimon, Atherosclerosis 1998, 136, 333-345.

### EMBODIMENTS

The application provides the following Embodiments:

Embodiment 1. A method of β- or γ-C(sp³)-H heteroarylation of aliphatic carboxylic acids, comprising treating an aliphatic carboxylic acid with a bidentate pyridine-pyridone ligand in the presence of a Pd(II) catalyst.

Embodiment 2. The method of Embodiment 1, wherein the method of β- or γ-C(sp³)-H heteroarylation of aliphatic carboxylic acids occurs according to the following reaction scheme:

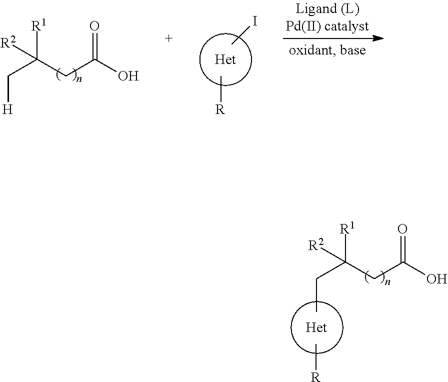

wherein:
Het is (C₅-C₆)heteroaryl;
R is H, halo, OH, (C₁-C₆)alkyl, halo (C₁-C₆)alkyl, halo (C₁-C₆)heteroalkyl, Ac, or —C(═O)H;
R¹ and R² are independently H or selected from the group consisting of (C₁-C₆)alkyl, (C₁-C₆)heteroalkyl, (C₃-C₇)cycloalkyl, (C₃-C₇)heterocycloalkyl, (C₆-C₁₀)aryl, (C₅-C₆)heteroaryl, (C₁-C₆)alkyl (C₃-C₇)cycloalkyl, (C₁-C₆)alkyl (C₃-C₇)heterocycloalkyl, (C₁-C₆)alkyl (C₆-C₁₀)aryl, (C₁-C₆)alkyl (C₅-C₆)heteroaryl, halo (C₁-C₆)alkyl, halo (C₁-C₆)heteroalkyl, halo (C₃-C₇)cycloalkyl, halo (C₃-C₇)heterocycloalkyl, halo (C₆-C₁₀)aryl, halo (C₅-C₆)heteroaryl, halo (C₁-C₆)alkyl (C₃-C₇)cycloalkyl, halo (C₁-C₆)alkyl (C₃-C₇)heterocycloalkyl, halo (C₁-C₆)alkyl (C₆-C₁₀)aryl, and halo (C₁-C₆)alkyl (C₅-C₆)heteroaryl, each independently and optionally substituted with one or more R′;
   each R′ is independently OH, halo, CN, (C₁-C₆)alkyl, halo (C₁-C₆)alkyl, (C₁-C₆)heteroalkyl, (C₃-C₇)cycloalkyl, (C₃-C₇)heterocycloalkyl, NH₂, or —S(═O)₂Me; and
n is 0 or 1.

Embodiment 3. The method of Embodiment 2, wherein n is 0.

Embodiment 4. The method of Embodiment 2, wherein n is 1.

Embodiment 5. The method of any one of Embodiments 2-4, wherein Het is pyridinyl.

Embodiment 6. The method of any one of Embodiments 2-4, wherein Het is pyrazinyl.

Embodiment 7. The method of any one of Embodiments 2-4, wherein Het is furanyl.

Embodiment 8. The method of any one of Embodiments 2-4, wherein Het is thiophenyl.

Embodiment 9. The method of any one of Embodiments 2-8, wherein R is H.

Embodiment 10. The method of any one of Embodiments 2-8, wherein R is Me.

Embodiment 11. The method of any one of Embodiments 2-8, wherein R is Cl.

Embodiment 12. The method of any one of Embodiments 2-8, wherein R is F.

Embodiment 13. The method of any one of Embodiments 2-8, wherein R is Br.

Embodiment 14. The method of any one of Embodiments 2-8, wherein R is CF₃.

Embodiment 15. The method of any one of Embodiments 2-8, wherein R is OMe.

Embodiment 16. The method of any one of Embodiments 2-8, wherein R is —C(═O)H or Ac.

Embodiment 17. The method of any one of Embodiments 2-16, wherein R¹ is H.

Embodiment 18. The method of any one of Embodiments 2-16, wherein R¹ is (C₁-C₆)alkyl.

Embodiment 19. The method of Embodiment 18, wherein R¹ is Me.

Embodiment 20. The method of Embodiment 18, wherein R¹ is Et.

Embodiment 21. The method of Embodiment 18, wherein R¹ is Pr.

Embodiment 22. The method of Embodiment 18, wherein R¹ is iPr.

Embodiment 23. The method of Embodiment 18, wherein R¹ is Bu.

Embodiment 24. The method of any one of Embodiments 2-16, wherein R is (C₁-C₆)heteroalkyl.

Embodiment 25. The method of any one of Embodiments 2-16, wherein R¹ is (C₆-C₁₀)aryl optionally substituted with one or more R′.

Embodiment 26. The method of Embodiment 25, wherein R¹ is Ph optionally substituted with one or more R′.

Embodiment 27. The method of any one of Embodiments 2-16, wherein R¹ is (C₁-C₆)alkyl (C₆-C₁₀)aryl optionally substituted with one or more R′.

Embodiment 28. The method of Embodiment 27, wherein R¹ is Bn optionally substituted with one or more R′.

Embodiment 29. The method of any one of Embodiments 25-18, wherein R′ is Me or halo.

Embodiment 30. The method of any one of Embodiments 2-29, wherein R² is H.

Embodiment 31. The method of any one of Embodiments 2-29, wherein R² is (C₁-C₆)alkyl.

Embodiment 32. The method of Embodiment 31, wherein R² is Me.

Embodiment 33. The method of Embodiment 31, wherein R² is Et.

Embodiment 34. The method of Embodiment 31, wherein R² is Pr.

Embodiment 35. The method of Embodiment 31, wherein R² is iPr.

Embodiment 36. The method of Embodiment 31, wherein R² is Bu.

Embodiment 37. The method of any one of Embodiments 2-29, wherein R² is (C₁-C₆)heteroalkyl.

Embodiment 38. The method of any one of Embodiments 2-29, wherein R² is (C₆-C₁₀)aryl optionally substituted with one or more R′.

Embodiment 39. The method of Embodiment 38, wherein R² is Ph optionally substituted with one or more R′.

Embodiment 40. The method of any one of Embodiments 2-29, wherein R² is (C₁-C₆)alkyl (C₆-C₁₀)aryl optionally substituted with one or more R′.

Embodiment 41. The method of Embodiment 40, wherein R² is Bn optionally substituted with one or more R′.

Embodiment 42. The method of any one of Embodiments 2-29, wherein R² is halo (C₁-C₆)alkyl (C₆-C₁₀)aryl.

Embodiment 43. The method of Embodiment 2, wherein R² and R³ together form (C₃-C₇)cycloalkyl.

Embodiment 44. The method of Embodiment 2, wherein R² and R³ together form cyclopropyl.

Embodiment 45. The method of Embodiment 2, wherein R² and R³ together form cyclobutyl.

Embodiment 46. The method of Embodiment 6, wherein R² is (C₃-C₆)cycloalkyl.

Embodiment 47. The method of any one of Embodiments 2-46, wherein L is selected from the group consisting of:

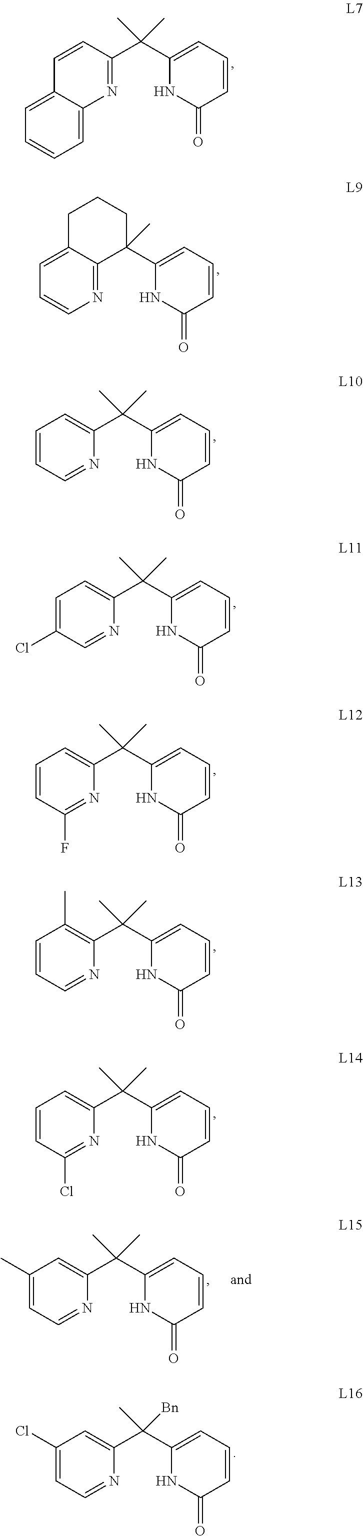

Embodiment 48. The method of Embodiment 47, wherein Lis L11.

Embodiment 49. The method of Embodiment 2, wherein the method of β- or γ-C(sp³)-H heteroarylation of aliphatic carboxylic acids occurs according to the following reaction scheme:

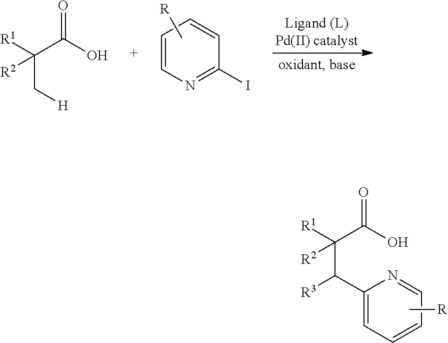

wherein:
R³ is H;
   or R² and R³ together form (C₃-C₄)cycloalkyl.

Embodiment 50. The method of Embodiment 2, wherein the method of β- or γ-C(sp³)-H heteroarylation of aliphatic carboxylic acids occurs according to the following reaction scheme:

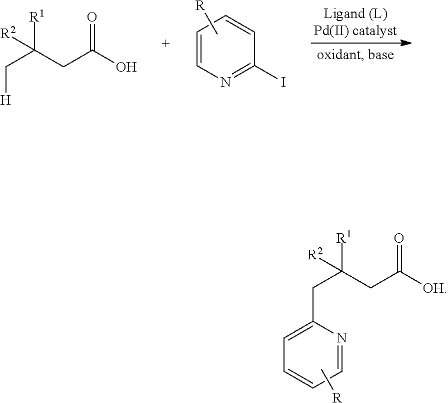

Embodiment 51. The method of either Embodiment 49 or Embodiment 50, wherein L is L11.

Embodiment 52. The method of any one of Embodiments 49-51, wherein R¹ is (C₁-C₆)alkyl.

Embodiment 53. The method of any one of Embodiments 49-52, wherein R² is (C₁-C₆)alkyl.

Embodiment 54. The method of any one of Embodiments 49-53, wherein R is H.

Embodiment 55. The method of any one of Embodiments 49-53, wherein R is Me.

Embodiment 56. The method of any one of Embodiments 49-53, wherein R is Cl.

Embodiment 57. The method of any one of Embodiments 49-53, wherein R is F.

Embodiment 58. The method of any one of Embodiments 49-53, wherein R is Br.

Embodiment 59. The method of any one of Embodiments 49-53, wherein R is CF₃.

Embodiment 60. The method of any one of Embodiments 49-53, wherein R is OMe.

Embodiment 61. The method any one of Embodiments 2-60, wherein the Pd(II) catalyst is Pd(OAc)₂.

Embodiment 62. The method any one of Embodiments 2-61, wherein the oxidant is Ag₂CO₃.

Embodiment 63. The method any one of Embodiments 2-62, wherein the base is KH₂PO₄.

Embodiment 64. The method any one of Embodiments 2-63, wherein the solvent is HFIP.

Embodiment 65. The method any one of Embodiments 2-64, wherein the concentration is approximately 1.0 M.

Embodiment 66. The method of Embodiment 2, wherein the method of β- or γ-C(sp³)-H heteroarylation of aliphatic carboxylic acids occurs according to the following reaction scheme:

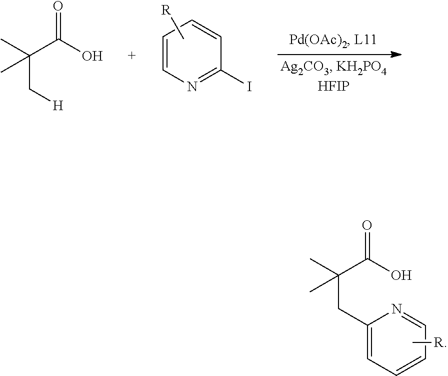

Embodiment 67. The method of Embodiment 2, wherein the method of β- or γ-C(sp³)-H heteroarylation of aliphatic carboxylic acids occurs according to the following reaction scheme:

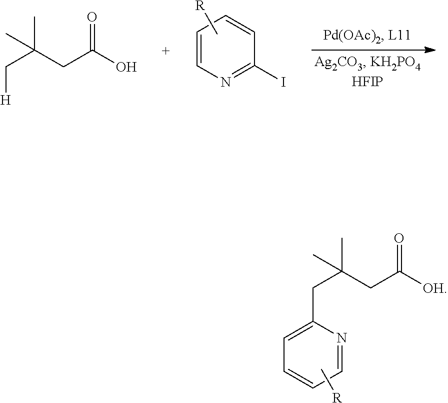

Embodiment 68. The method of any one of Embodiments 2-67, wherein the reaction temperature is between approximately 110-140° C.

Embodiment 69. The method of Embodiment 68, wherein the reaction temperature is approximately 120° C.

Embodiment 70. Any method of β- or γ-C(sp³)-H heteroarylation of aliphatic carboxylic acids as disclosed herein.

### Definitions

The phrase “a” or “an” entity as used herein refers to one or more of that entity; for example, a compound refers to one or more compounds or at least one compound. As such, the terms “a” (or “an”), “one or more”, and “at least one” can be used interchangeably herein.

The phrase “as defined herein above” refers to the broadest definition for each group as provided in the Summary of the Invention, the Detailed Description of the Invention, the Experimentals, or the broadest claim. In all other embodiments provided below, substituents which can be present in each embodiment and which are not explicitly defined retain the broadest definition provided in the Summary of the Invention.

As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least”. When used in the context of a process, the term “comprising” means that the process includes at least the recited steps, but may include additional steps. When used in the context of a compound or composition, the term “comprising” means that the compound or composition includes at least the recited features or components, but may also include additional features or components.

As used herein, unless specifically indicated otherwise, the word “or” is used in the “inclusive” sense of “and/or” and not the “exclusive” sense of “either/or”.

The term “independently” is used herein to indicate that a variable is applied in any one instance without regard to the presence or absence of a variable having that same or a different definition within the same compound. Thus, in a compound in which “R” appears twice and is defined as “independently selected from” means that each instance of that R group is separately identified as one member of the set which follows in the definition of that R group. For example, “each R¹ and R² is independently selected from carbon and nitrogen” means that both R¹ and R² can be carbon, both R¹ and R² can be nitrogen, or R¹ or R² can be carbon and the other nitrogen or vice versa.

When any variable occurs more than one time in any moiety or formula depicting and describing compounds employed or claimed in the present invention, its definition on each occurrence is independent of its definition at every other occurrence. Also, combinations of substituents and/or variables are permissible only if such compounds result in stable compounds.

The symbols “*” at the end of a bond or a line drawn through a bond or “˜˜˜˜” drawn through a bond each refer to the point of attachment of a functional group or other chemical moiety to the rest of the molecule of which it is a part.

A bond drawn into ring system (as opposed to connected at a distinct vertex) indicates that the bond may be attached to any of the suitable ring atoms.

The term “optional” or “optionally” as used herein means that a subsequently described event or circumstance may, but need not, occur, and that the description includes instances where the event or circumstance occurs and instances in which it does not. For example, “optionally substituted” means that the “optionally substituted” moiety may incorporate a hydrogen or a substituent.

The phrase “optional bond” means that the bond may or may not be present, and that the description includes single, double, or triple bonds. If a substituent is designated to be a “bond” or “absent”, the atoms linked to the substituents are then directly connected.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

Certain compounds disclosed herein may exhibit tautomerism. Tautomeric compounds can exist as two or more interconvertable species. Prototropic tautomers result from the migration of a covalently bonded hydrogen atom between two atoms. Tautomers generally exist in equilibrium and attempts to isolate an individual tautomers usually produce a mixture whose chemical and physical properties are consistent with a mixture of compounds. The position of the equilibrium is dependent on chemical features within the molecule. For example, in many aliphatic aldehydes and ketones, such as acetaldehyde, the keto form predominates while; in phenols, the enol form predominates. Common prototropic tautomers include keto/enol (—C(═O)—CH—⇄—C(—OH)═CH—), amide/imidic acid (—C(═O)—NH—⇄—C(—OH)═N—) and amidine (—C(═NR)—NH—⇄—C(—NHR)═N—) tautomers. The latter two are particularly common in heteroaryl and heterocyclic rings and the present invention encompasses all tautomeric forms of the compounds.

Technical and scientific terms used herein have the meaning commonly understood by one of skill in the art to which the present invention pertains, unless otherwise defined. Reference is made herein to various methodologies and materials known to those of skill in the art. Standard reference works setting forth the general principles of pharmacology include Goodman and Gilman's The Pharmacological Basis of Therapeutics, 10^{th} Ed., McGraw Hill Companies Inc., New York (2001). Any suitable materials and/or methods known to those of skill can be utilized in carrying out the present invention. However, preferred materials and methods are described. Materials, reagents and the like to which reference are made in the following description and examples are obtainable from commercial sources, unless otherwise noted.

The definitions described herein may be appended to form chemically-relevant combinations, such as “heteroalkylaryl,” “haloalkylheteroaryl,” “arylalkylheterocyclyl,” “alkylcarbonyl,” “alkoxyalkyl,” and the like. When the term “alkyl” is used as a suffix following another term, as in “phenylalkyl,” or “hydroxyalkyl,” this is intended to refer to an alkyl group, as defined above, being substituted with one to two substituents selected from the other specifically-named group. Thus, for example, “phenylalkyl” refers to an alkyl group having one to two phenyl substituents, and thus includes benzyl, phenylethyl, and biphenyl. An “alkylaminoalkyl” is an alkyl group having one to two alkylamino substituents. “Hydroxyalkyl” includes 2-hydroxyethyl, 2-hydroxypropyl, 1-(hydroxymethyl)-2-methylpropyl, 2-hydroxybutyl, 2,3-dihydroxybutyl, 2-(hydroxymethyl), 3-hydroxypropyl, and so forth. Accordingly, as used herein, the term “hydroxyalkyl” is used to define a subset of heteroalkyl groups defined below. The term -(ar)alkyl refers to either an unsubstituted alkyl or an aralkyl group. The term (hetero)aryl or (het)aryl refers to either an aryl or a heteroaryl group.

The term “acyl” as used herein denotes a group of formula —C(═O)R wherein R is hydrogen or lower alkyl as defined herein. The term or “alkylcarbonyl” as used herein denotes a group of formula C(═O)R wherein R is alkyl as defined herein. The term C₁₋₆ acyl refers to a group —C(═O)R contain 6 carbon atoms. The term “arylcarbonyl” as used herein means a group of formula C(═O)R wherein R is an aryl group; the term “benzoyl” as used herein an “arylcarbonyl” group wherein R is phenyl.

The term “alkyl” as used herein denotes an unbranched or branched chain, saturated, monovalent hydrocarbon residue containing 1 to 12 carbon atoms. The term “lower alkyl” or “C₁-C₆ alkyl” as used herein denotes a straight or branched chain hydrocarbon residue containing 1 to 6 carbon atoms. “C₁₋₁₂ alkyl” as used herein refers to an alkyl composed of 1 to 12 carbons. Examples of alkyl groups include, but are not limited to, lower alkyl groups include methyl, ethyl, propyl, i-propyl, n-butyl, i-butyl, t-butyl or pentyl, isopentyl, neopentyl, hexyl, heptyl, and octyl.

When the term “alkyl” is used as a suffix following another term, as in “phenylalkyl,” or “hydroxyalkyl,” this is intended to refer to an alkyl group, as defined above, being substituted with one to two substituents selected from the other specifically-named group. Thus, for example, “phenylalkyl” denotes the radical R′R″—, wherein R′ is a phenyl radical, and R″ is an alkylene radical as defined herein with the understanding that the attachment point of the phenylalkyl moiety will be on the alkylene radical. Examples of arylalkyl radicals include, but are not limited to, benzyl, phenylethyl, 3-phenylpropyl. The terms “arylalkyl” or “aralkyl” are interpreted similarly except R′ is an aryl radical. The terms “(het)arylalkyl” or “(het)aralkyl” are interpreted similarly except R′ is optionally an aryl or a heteroaryl radical.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

“Alkyl” refers to a radical of a straight-chain or branched saturated hydrocarbon group having from 1 to 20 carbon atoms (“C₁₋₂₀ alkyl”). In some embodiments, an alkyl group has 1 to 15 carbon atoms (“C₁₋₁₅ alkyl”). In some embodiments, an alkyl group has 1 to 14 carbon atoms (“C₁₋₁₄ alkyl”). In some embodiments, an alkyl group has 1 to 13 carbon atoms (“C₁₋₁₃ alkyl”). In some embodiments, an alkyl group has 1 to 12 carbon atoms (“C₁₋₁₂ alkyl”). In some embodiments, an alkyl group has 1 to 11 carbon atoms (“C₁₋₁₁ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like.

“Alkenyl” or “olefin” refers to a radical of a straight-chain or branched hydrocarbon group having from 2 to 10 carbon atoms and 1, 2, 3, or 4 carbon-carbon double bonds (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like.

“Alkynyl” refers to a radical of a straight-chain or branched hydrocarbon group having from 2 to 10 carbon atoms and one or more carbon-carbon triple bonds (e.g., 1, 2, 3, or 4 triple bonds) (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like.

The terms “haloalkyl” or “halo-lower alkyl” or “lower haloalkyl” refers to a straight or branched chain hydrocarbon residue containing 1 to 6 carbon atoms wherein one or more carbon atoms are substituted with one or more halogen atoms.

The term “alkylene” or “alkylenyl” as used herein denotes a divalent saturated linear hydrocarbon radical of 1 to 10 carbon atoms (e.g., (CH₂)ₙ) or a branched saturated divalent hydrocarbon radical of 2 to 10 carbon atoms (e.g., —CHMe- or —CH₂CH(i-Pr)CH₂—), unless otherwise indicated. Except in the case of methylene, the open valences of an alkylene group are not attached to the same atom. Examples of alkylene radicals include, but are not limited to, methylene, ethylene, propylene, 2-methyl-propylene, 1,1-dimethyl-ethylene, butylene, 2-ethylbutylene.

The term “alkoxy” as used herein means an —O-alkyl group, wherein alkyl is as defined above such as methoxy, ethoxy, n-propyloxy, i-propyloxy, n-butyloxy, i-butyloxy, t-butyloxy, pentyloxy, hexyloxy, including their isomers. “Lower alkoxy” as used herein denotes an alkoxy group with a “lower alkyl” group as previously defined. “C₁₋₁₀ alkoxy” as used herein refers to an —O-alkyl wherein alkyl is C₁₋₁₀.

The term “hydroxyalkyl” as used herein denotes an alkyl radical as herein defined wherein one to three hydrogen atoms on different carbon atoms is/are replaced by hydroxyl groups.

The terms “alkylsulfonyl” and “arylsulfonyl” as used herein refers to a group of formula —S(═O)₂R wherein R is alkyl or aryl respectively and alkyl and aryl are as defined herein. The term “heteroalkylsulfonyl” as used herein refers herein denotes a group of formula —S(═O)₂R wherein R is “heteroalkyl” as defined herein.

The terms “alkylsulfonylamino” and “arylsulfonylamino” as used herein refers to a group of formula —NR'S(═O)₂R wherein R is alkyl or aryl respectively, R′ is hydrogen or C₁₋₃ alkyl, and alkyl and aryl are as defined herein.

The term “cycloalkyl” as used herein refers to a saturated carbocyclic ring containing 3 to 8 carbon atoms, i.e. cyclopropyl, cyclobutyl, cyclopentyl, cyclohexyl, cycloheptyl or cyclooctyl. “C₃₋₇ cycloalkyl” as used herein refers to an cycloalkyl composed of 3 to 7 carbons in the carbocyclic ring.

The term carboxy-alkyl as used herein refers to an alkyl moiety wherein one, hydrogen atom has been replaced with a carboxyl with the understanding that the point of attachment of the heteroalkyl radical is through a carbon atom. The term “carboxy” or “carboxyl” refers to a —CO₂H moiety.

The term “heteroaryl” or “heteroaromatic” as used herein means a monocyclic or bicyclic radical of 5 to 12 ring atoms having at least one aromatic ring containing four to eight atoms per ring, incorporating one or more N, O, or S heteroatoms, the remaining ring atoms being carbon, with the understanding that the attachment point of the heteroaryl radical will be on an aromatic ring. As well known to those skilled in the art, heteroaryl rings have less aromatic character than their all-carbon counter parts. Thus, for the purposes of the invention, a heteroaryl group need only have some degree of aromatic character. Examples of heteroaryl moieties include monocyclic aromatic heterocycles having 5 to 6 ring atoms and 1 to 3 heteroatoms include, but is not limited to, pyridinyl, pyrimidinyl, pyrazinyl, pyrrolyl, pyrazolyl, imidazolyl, oxazol, isoxazole, thiazole, isothiazole, triazoline, thiadiazole and oxadiaxoline which can optionally be substituted with one or more, preferably one or two substituents selected from hydroxy, cyano, alkyl, alkoxy, thio, lower haloalkoxy, alkylthio, halo, lower haloalkyl, alkylsulfinyl, alkylsulfonyl, halogen, amino, alkylamino, dialkylamino, aminoalkyl, alkylaminoalkyl, and dialkylaminoalkyl, nitro, alkoxycarbonyl and carbamoyl, alkylcarbamoyl, dialkylcarbamoyl, arylcarbamoyl, alkylcarbonylamino and arylcarbonylamino. Examples of bicyclic moieties include, but are not limited to, quinolinyl, isoquinolinyl, benzofuryl, benzothiophenyl, benzoxazole, benzisoxazole, benzothiazole and benzisothiazole. Bicyclic moieties can be optionally substituted on either ring; however the point of attachment is on a ring containing a heteroatom.

The term “heterocyclyl”, “heterocycloalkyl” or “heterocycle” as used herein denotes a monovalent saturated cyclic radical, consisting of one or more rings, preferably one to two rings, including spirocyclic ring systems, of three to eight atoms per ring, incorporating one or more ring heteroatoms (chosen from N, O or S(O)₀₋₂), and which can optionally be independently substituted with one or more, preferably one or two substituents selected from hydroxy, oxo, cyano, lower alkyl, lower alkoxy, lower haloalkoxy, alkylthio, halo, lower haloalkyl, hydroxyalkyl, nitro, alkoxycarbonyl, amino, alkylamino, alkylsulfonyl, arylsulfonyl, alkylaminosulfonyl, arylaminosulfonyl, alkylsulfonylamino, arylsulfonylamino, alkylaminocarbonyl, arylaminocarbonyl, alkylcarbonylamino, arylcarbonylamino, unless otherwise indicated. Examples of heterocyclic radicals include, but are not limited to, azetidinyl, pyrrolidinyl, hexahydroazepinyl, oxetanyl, tetrahydrofuranyl, tetrahydrothiophenyl, oxazolidinyl, thiazolidinyl, isoxazolidinyl, morpholinyl, piperazinyl, piperidinyl, tetrahydropyranyl, thiomorpholinyl, quinuclidinyl and imidazolinyl.

“Heterocyclyl” or “heterocyclic” refers to a group or radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include, without limitation, azirdinyl, oxiranyl, and thiiranyl. Exemplary 4-membered heterocyclyl groups containing 1 heteroatom include, without limitation, azetidinyl, oxetanyl and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, and dioxanyl. Exemplary 6-membered heterocyclyl groups containing 3 heteroatoms include, without limitation, triazinanyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo-[2,3-b]pyridinyl, 4,5,6,7-tetrahydrofuro[3,2-c]pyridinyl, 4,5,6,7-tetrahydrothieno[3,2-b] pyridinyl, 1,2,3,4-tetrahydro-1,6-naphthyridinyl, and the like.

“Aryl” refers to a radical of a monocyclic or polycyclic (e.g., bicyclic or tricyclic) 4n+2 aromatic ring system (e.g., having 6, 10, or 14 pi electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl (α-naphthyl) and 2-naphthyl (β-naphthyl)). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system.

“Heteroaryl” refers to a radical of a 5-14 membered monocyclic or polycyclic (e.g., bicyclic, tricyclic) 4n+2 aromatic ring system (e.g., having 6, 10, or 14 pi electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur.

Exemplary 5-membered heteroaryl groups containing 1 heteroatom include, without limitation, pyrrolyl, furanyl and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl and phenazinyl.

“Saturated” refers to a ring moiety that does not contain a double or triple bond, i.e., the ring contains all single bonds.

Alkyl, cycloalkyl, heterocyclyl, aryl, and heteroaryl groups may be optionally substituted. Optionally substituted refers to a group which may be substituted or unsubstituted. In general, the term “substituted” means that at least one hydrogen present on a group is replaced with a non-hydrogen substituent, and which upon substitution results in a stable compound, e.g., a compound which does not spontaneously undergo transformation such as by rearrangement, cyclization, elimination, or other reaction. Heteroatoms such as nitrogen, oxygen, and sulfur may have hydrogen substituents and/or non-hydrogen substituents which satisfy the valencies of the heteroatoms and results in the formation of a stable compound.

Exemplary non-hydrogen substituents wherein a moiety is “optionally substituted” as used herein means the moiety may be substituted with any additional moiety selected from, but not limited to, the group consisting of halogen, —CN, —NO₂, —N₃, —SO₂H, —SO₃H, —OH, —OR^{aa}, —N(R^{bb})₂, —N(OR^{cc})R^{bb}, —SH, —SR^{aa}, —C(═O)R^{aa}, —CO₂H, —CHO, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O) NR^{bb}SO₂R^{aa}, —NR^{bb}SO₂R^{aa}, —SO₂N(R^{bb})₂, —SO₂R^{aa}, —S(═O)R^{aa}, —OS(═O)R^{aa}, —B(OR)², C₁₋₁₀ alkyl, C₂₋₁₀ alkenyl, C₂₋₁₀ alkynyl, C₃₋₁₄ carbocyclyl, 3- to 14-membered heterocyclyl, C₆₋₁₄ aryl, and 5- to 14-membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups, or two geminal hydrogens on a carbon atom are replaced with the group-O; each instance of R^{aa} is, independently, selected from the group consisting of C₁₋₁₀ alkyl, C₁₋₁₀ perhaloalkyl, C₂₋₁₀ alkenyl, C₂₋₁₀ alkynyl, C₃₋₁₄ carbocyclyl, 3- to 14-membered heterocyclyl, C₆₋₁₄ aryl, and 5- to 14-membered heteroaryl, or two R^{aa} groups are joined to form a 3- to 14-membered heterocyclyl or 5- to 14-membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups; each instance of R^{bb} is, independently, selected from the group consisting of hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —SO₂N(R^{cc})₂, —SOR^{aa}, C₁₋₁₀ alkyl, C₁₋₁₀ perhaloalkyl, C₂₋₁₀ alkenyl, C₂₋₁₀ alkynyl, C₃₋₁₄ carbocyclyl, 3- to 14-membered heterocyclyl, C₆₋₁₄ aryl, and 5- to 14-membered heteroaryl, or two R^{bb} groups are joined to form a 3- to 14-membered heterocyclyl or 5- to 14-membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups; each instance of R^{cc} is, independently, selected from the group consisting of hydrogen, C₁₋₁₀ alkyl, C₁₋₁₀ perhaloalkyl, C₂₋₁₀ alkenyl, C₂₋₁₀ alkynyl, C₃₋₁₄ carbocyclyl, 3- to 14-membered heterocyclyl, C₆₋₁₄ aryl, and 5- to 14-membered heteroaryl, or two R^{cc} groups are joined to form a 3- to 14-membered heterocyclyl or 5- to 14-membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups; and each instance of R^{dd} is, independently, selected from the group consisting of halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(OC₁₋₆ alkyl)(C₁₋₆ alkyl), —N(OH)(C₁₋₆ alkyl), —NH(OH), —SH, —SC₁₋₆ alkyl, —C(═O)(C₁₋₆ alkyl), —CO₂H, —CO₂ (C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl) C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —B(OH)₂, —B(OC₁₋₆ alkyl)₂, C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3- to 10-membered heterocyclyl, and 5- to 10-membered heteroaryl; or two geminal R^{dd} substituents on a carbon atom may be joined to form ═O.

“Halo” or “halogen” refers to fluorine (fluoro, —F), chlorine (chloro, —Cl), bromine (bromo, —Br), or iodine (iodo, —I).

As used herein, the term “composition” is intended to encompass a product comprising the specified ingredients, as well as any product which results, directly or indirectly, from combination of the specified ingredients.

“Salt” includes any and all salts. “Pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts include those derived from inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

Unless otherwise indicated, compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC). Compounds described herein can be in the form of individual isomers substantially free of other isomers, and alternatively, as mixtures of various isomers.

Unless otherwise stated, structures depicted herein are also meant to include compounds that differ only in the presence of one or more isotopically enriched atoms. For example, compounds having the present structures except for the replacement of hydrogen by deuterium or tritium, replacement of ¹⁹F with ¹⁸F, replacement of a carbon by a ¹³C- or ¹⁴C-enriched carbon, and/or replacement of an oxygen atom with ¹⁸O, are within the scope of the disclosure. Other examples of isotopes include ¹⁵N, ¹⁸O, ¹⁷O, ³¹P, ³²P, ³⁵S, ¹⁸F, ³⁶Cl and ¹²³I. Compounds with such isotopically enriched atoms are useful, for example, as analytical tools or probes in biological assays.

Certain isotopically-labelled compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability.

Certain isotopically-labelled compounds of Formula (I) can be useful for medical imaging purposes, for example, those labeled with positron-emitting isotopes like ¹¹C or ¹⁸F can be useful for application in Positron Emission Tomography (PET) and those labeled with gamma ray emitting isotopes like ¹²³I can be useful for application in Single Photon Emission Computed Tomography (SPECT). Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Further, substitution with heavier isotopes such as deuterium (i.e., ²H) may afford certain therapeutic advantages resulting from greater metabolic stability (e.g., increased in vivo half-life or reduced dosage requirements), and hence, may be preferred in some circumstances. Additionally, isotopic substitution at a site where epimerization occurs may slow or reduce the epimerization process and thereby retain the more active or efficacious form of the compound for a longer period of time. Isotopically labeled compounds of Formula (I), in particular those containing isotopes with longer half-lives (t_{1/2}>1 day), can generally be prepared by following procedures analogous to those disclosed in the Schemes and/or in the Examples herein below, by substituting an appropriate isotopically labeled reagent for a non-isotopically labeled reagent.

If there is a discrepancy between a depicted structure and a name given to that structure, then the depicted structure controls. Additionally, if the stereochemistry of a structure or a portion of a structure is not indicated with, for example, bold or dashed lines, the structure or portion of the structure is to be interpreted as encompassing all stereoisomers of it. In some cases, however, where more than one chiral center exists, the structures and names may be represented as single enantiomers to help describe the relative stereochemistry. Those skilled in the art of organic synthesis will know if the compounds are prepared as single enantiomers from the methods used to prepare them.

### Examples

### Abbreviations

Commonly used abbreviations include: acetyl (Ac), azo-bis-isobutyrylnitrile (AIBN), atmospheres (Atm), 9-borabicyclo[3.3.1]nonane (9-BBN or BBN), tert-butoxycarbonyl (Boc), di-tert-butyl pyrocarbonate or boc anhydride (BOC₂O), benzyl (Bn), butyl (Bu), Chemical Abstracts Registration Number (CASRN), benzyloxycarbonyl (CBZ or Z), carbonyl diimidazole (CDI), 1,4-diazabicyclo[2.2.2]octane (DABCO), diethylaminosulfur trifluoride (DAST), dibenzylideneacetone (dba), 1,5-diazabicyclo[4.3.0]non-5-ene (DBN), 1,8-diazabicyclo[5.4.0]undec-7-ene (DBU), N,N′-dicyclohexylcarbodiimide (DCC), 1,2-dichloroethane (DCE), dichloromethane (DCM), diethyl azodicarboxylate (DEAD), di-iso-propylazodicarboxylate (DIAD), di-iso-butylaluminumhydride (DIBAL or DIBAL-H), 1,3-Diisopropylcarbodiimide (DIC), di-iso-propylethylamine (DIPEA), N,N-dimethyl acetamide (DMA), 4-N,N-dimethylaminopyridine (DMAP), N,N-dimethylformamide (DMF), dimethyl sulfoxide (DMSO), 1,1′-bis-(diphenylphosphino) ethane (dppe), 1,1′-bis-(diphenylphosphino) ferrocene (dppf), 1-(3-dimethylaminopropyl)-3-ethylcarbodiimide hydrochloride (EDCI), ethyl (Et), ethyl acetate (EtOAc), ethanol (EtOH), 2-ethoxy-2H-quinoline-1-carboxylic acid ethyl ester (EEDQ), diethyl ether (Et₂O), O-(7-azabenzotriazole-1-yl)-N, N,N′N′-tetramethyluronium hexafluorophosphate acetic acid (HATU), acetic acid (HOAc), 1-N-hydroxybenzotriazole (HOBt), high pressure liquid chromatography (HPLC), iso-propanol (IPA), lithium hexamethyl disilazane (LiHMDS), methanol (MeOH), melting point (mp), MeSO₂-(mesyl or Ms), methyl (Me), acetonitrile (MeCN), m-chloroperbenzoic acid (MCPBA), mass spectrum (ms), methyl 1-butyl ether (MTBE), N-bromosuccinimide (NBS), N-carboxyanhydride (NCA), N-chlorosuccinimide (NCS), N-methylmorpholine (NMM), N-methylpyrrolidone (NMP), pyridinium chlorochromate (PCC), pyridinium dichromate (PDC), phenyl (Ph), propyl (Pr), iso-propyl (i-Pr), pounds per square inch (psi), pyridine (pyr), room temperature (rt or RT), tert-butyldimethylsilyl or 1-BuMe₂Si (TBDMS), triethylamine (TEA or Et₃N), 2,2,6,6-tetramethylpiperidine 1-oxyl (TEMPO), triflate or CF₃SO₂-(Tf), trifluoroacetic acid (TFA), 1,1′-bis-2,2,6,6-tetramethylheptane-2,6-dione (TMHD), O-benzotriazol-1-yl-N,N,N′,N′-tetramethyluronium tetrafluoroborate (TBTU), thin layer chromatography (TLC), tetrahydrofuran (THF), trimethylsilyl or Me₃Si (TMS), p-toluenesulfonic acid monohydrate (TsOH or pTsOH), 4-Me-C₆H₄SO₂— or tosyl (Ts), N-urethane-N-carboxyanhydride (UNCA). Conventional nomenclature including the prefixes normal (n), iso (i-), secondary (sec-), tertiary (tert-) and neo have their customary meaning when used with an alkyl moiety. (J. Rigaudy and D. P. Klesney, Nomenclature in Organic Chemistry, IUPAC 1979 Pergamon Press, Oxford.).

### General Information

Pd(OAc)₂ was purchased from Sigma-Aldrich. Ag₂CO₃ were purchased from Strem. Solvents were obtained from Sigma-Aldrich, Acros and Oakwood, and used directly without further purification. Heterocycle iodides were obtained from the commercial sources. Carboxylic acids were obtained from the commercial sources or synthesized following literature procedures. Other reagents were purchased at the highest commercial quality and used without further purification, unless otherwise stated. Analytical thin layer chromatography was performed on 0.25 mm silica gel 60-F254. Visualization was carried out with UV light. 1H NMR spectra were recorded on Bruker DRX-600 instrument. Chemical shifts were quoted in parts per million (ppm) referenced to 0.00 ppm for TMS. The following abbreviations (or combinations thereof) were used to explain multiplicities: s=singlet, d=doublet, t=triplet, q=quartet, m=multiplet, br=broad. Coupling constants, J, were reported in Hertz unit (Hz). ¹³C NMR spectra were recorded on Bruker DRX-600 was fully decoupled by broad band proton decoupling. Chemical shifts were reported in ppm referenced to the centre line of a triplet at 77.16 ppm of CDCl₃. ¹⁹F NMR spectra were recorded on Bruker AMX-400 instrument (376 MHz) and were fully decoupled by broad band proton decoupling. Chemical shifts were reported in ppm referenced to the centre line of a triplet at 77.0 ppm of chloroform-d or the centre line of a heptet at 49.0 ppm. Column chromatography was performed using E. Merck silica (60, particle size 0.043-0.063 mm), and preparative thin layer chromatography (pTLC) was performed on Merck silica plates (60F-254). High-resolution mass spectra (HRMS) were recorded on an Agilent Mass spectrometer using ESI-TOF (electrospray ionization-time of flight).

N/B: All Carboxylic Acids were Obtained from the Commercial Sources or Synthesized Following Literature Procedures.⁴⁻⁷ all of the Substrates have been Previously Reported.

General procedure for aza-heteroarylation of carboxylic acids:

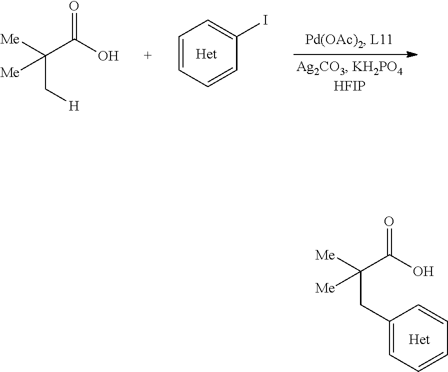 [SMILES: CC(C)(C)C(=O)O ; Ic1ccccc1 ; CC(C)(Cc1ccccc1)C(=O)O]

Carboxylic acid (0.1 mmol), Iodoheterocycle (0.2 mmol), Pd(OAc)₂ (10 mol %), Ligand (10 mol %), Ag₂CO₃ (2.0 equiv.), KH₂PO₄ (3.0 equiv.) and HFIP (0.1 ml) were added to a reaction vial (8 ml). The vial was capped under air and closed tightly. Then the reaction mixture was stirred at 120° C. for 24 hours. After cooling to room temperature, the mixture was acidified with AcOH (5.0 equiv.) and filtered through a pad of celite with acetone as the eluent to remove the insoluble precipitate. The resulting solution was concentrated and purified by preparative thin-layer chromatography to afford the desired product.

### Substrate Scope

### Scope of Heterocycle Iodides for β-C(sp³)-H Heteroarylation

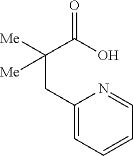 [SMILES: CC(C)(Cc1ccccn1)C(=O)O]

### 2,2-Dimethyl-3-(pyridin-2-yl)propanoic Acid (3a)

Following General Procedures. Purification by pTLC (50% EA/hexanes, Rf=0.35) afforded the title compound as colourless oil (12.5 mg, 0.07 mmol). ¹H NMR (600 MHz, CDCl₃) δ 8.55 (d, J=4.5 Hz, 1H), 7.80 (t, J=7.7 Hz, 1H), 7.36-7.31 (m, 1H), 7.30 (d, J=7.7 Hz, 1H), 3.11 (s, 2H), 1.27 (s, 6H). ¹³C NMR (151 MHz, CDCl₃) δ 178.82, 157.70, 146.55, 138.65, 125.54, 122.44, 45.08, 42.85, 26.67.

HRMS (ESI-TOF) Calcd for C₁₀H₁₄NO₂⁺[M+H]⁺: 180.1025; found: 180.1022.

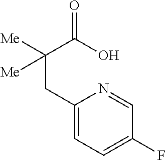 [SMILES: CC(C)(Cc1ccc(F)cn1)C(=O)O]

### 3-(5-Fluoropyridin-2-yl)-2,2-dimethylpropanoic Acid (3b)

Following General Procedure. Purification by pTLC (35% EA/hexanes, Rf=0.30) afforded the title compound as colourless oil (11.6 mg, 0.06 mmol). ¹H NMR (600 MHz, CDCl₃) δ 8.48 (d, J=2.7 Hz, 1H), 7.43 (td, J=8.3, 2.9 Hz, 1H), 7.24 (dd, J=8.6, 4.4 Hz, 1H), 3.11 (s, 2H), 1.28 (s, 6H). ¹³C NMR (151 MHz, CDCl₃) δ 179.68, 158.59 (d, J_{CF}=255.2 Hz), 153.95 (d, J_{CF}=4.2 Hz), 136.38 (d, J_{CF}=24.9 Hz), 125.95 (d, J_{CF}=4.5 Hz), 124.31 (d, J_{CF}=18.3 Hz), 45.92, 42.93, 25.73. ¹⁹F NMR (376 MHz, CDCl₃) δ −131.47. HRMS (ESI-TOF) Calcd for C₁₀H₁₃FNO₂⁺ [M+H]⁺: 198.0930; found: 198.0932.

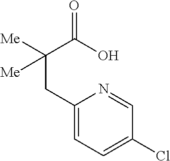 [SMILES: CC(C)(Cc1ccc(Cl)cn1)C(=O)O]

### 3-(5-Chloropyridin-2-yl)-2,2-dimethylpropanoic acid (3c)

Following General Procedure. Purification by pTLC (35% EA/hexanes, Rf=0.30) afforded the title compound as colourless oil (15.7 mg, 0.07 mmol). ¹H NMR (600 MHz, CDCl₃) δ 8.58 (s, 1H), 7.68 (d, J=8.1 Hz, 1H), 7.20 (d, J=8.3 Hz, 1H), 3.10 (s, 2H), 1.28 (s, 6H). ¹³C NMR (151 MHz, CDCl₃) δ 179.67, 156.12, 147.10, 137.08, 130.67, 125.80, 46.14, 42.92, 25.73.

HRMS (ESI-TOF) Calcd for C₁₀H₁₃ClNO₂⁺ [M+H]⁺: 214.0635; found: 214.0637.

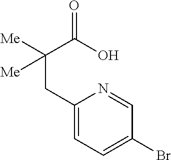 [SMILES: CC(C)(Cc1ccc(Br)cn1)C(=O)O]

### 3-(5-Bromopyridin-2-yl)-2,2-dimethylpropanoic Acid (3d)

Following General Procedure. Purification by pTLC (35% EA/hexanes, Rf=0.30) afforded the title compound as colourless oil (17.2 mg, 0.07 mmol). ¹H NMR (600 MHz, CDCl₃) δ 8.68 (s, 1H), 7.81 (d, J=8.3 Hz, 1H), 7.14 (d, J=8.3 Hz, 1H), 3.08 (s, 2H), 1.28 (s, 6H). ¹³C NMR (151 MHz, CDCl₃) δ 180.18, 156.49, 149.37, 139.82, 126.24, 119.03, 46.29, 42.88, 25.60.

HRMS (ESI-TOF) Calcd for C₁₀H₁₃BrNO₂⁺ [M+H]⁺: 258.0130; found: 258.0127.

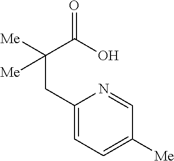 [SMILES: Cc1ccc(CC(C)(C)C(=O)O)nc1]

### 2,2-Dimethyl-3-(5-methylpyridin-2-yl)propanoic Acid (3e)

Following General Procedure. Purification by pTLC (45% EA/hexanes, Rf=0.35) afforded the title compound as colourless oil (12.0 mg, 0.06 mmol). ¹H NMR (600 MHz, CDCl₃) δ 8.36 (s, 1H), 7.60 (d, J=7.9 Hz, 1H), 7.18 (d, J=7.9 Hz, 1H), 3.05 (s, 2H), 2.39 (s, 3H), 1.25 (s, 6H). ¹³C NMR (151 MHz, CDCl₃) δ 179.15, 154.56, 146.70, 139.15, 132.18, 125.02, 44.81, 42.88, 26.54, 18.08.

HRMS (ESI-TOF) Calcd for C₁₁H₁₆NO₂⁺ [M+H]⁺: 194.1181; found: 194.1185.

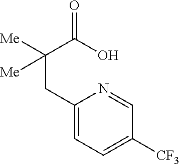 [SMILES: CC(C)(Cc1ccc(C(F)(F)F)cn1)C(=O)O]

### 2,2-Dimethyl-3-(5-(trifluoromethyl)pyridin-2-yl)propanoic Acid (3f)

Following General Procedure. Purification by pTLC (35% EA/hexanes, Rf=0.30) afforded the title compound as colourless oil (10.1 mg, 0.04 mmol). ¹H NMR (600 MHz, CDCl₃) δ 8.87 (s, 1H), 7.92 (dd, J=8.1, 2.0 Hz, 1H), 7.38 (d, J=8.1 Hz, 1H), 3.21 (s, 2H), 1.30 (s, 6H). ¹³C NMR (151 MHz, CDCl₃) δ 180.10, 162.13, 145.43, 134.05, 125.15 (q, J_{CF}=32.5 Hz), 124.68, 123.39 (q, J_{CF}=274.8 Hz), 46.93, 42.88, 25.65. ¹⁹F NMR (376 MHz, CDCl₃) δ −65.04.

HRMS (ESI-TOF) Calcd for C₁₁H₁₃F₃NO₂⁺ [M+H]⁺: 248.0898; found: 248.0900.

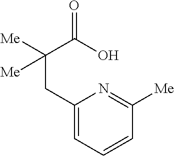 [SMILES: Cc1cccc(CC(C)(C)C(=O)O)n1]

### 2,2-Dimethyl-3-(6-methylpyridin-2-yl)propanoic Acid (3g)

Following General Procedure. Purification by pTLC (90% EA/hexanes, Rf=0.35) afforded the title compound as colourless oil (13.9 mg, 0.07 mmol). ¹H NMR (600 MHz, CDCl₃) δ 7.70 (t, J=7.7 Hz, 1H), 7.18 (d, J=7.8 Hz, 1H), 7.10 (d, J=7.7 Hz, 1H), 3.05 (s, 2H), 2.62 (s, 3H), 1.25 (s, 6H). ¹³C NMR (151 MHz, CDCl₃) δ 179.20, 156.76, 155.95, 139.16, 122.53, 122.34, 44.51, 42.88, 26.90, 22.81.

HRMS (ESI-TOF) Calcd for C₁₁H₁₆NO₂⁺ [M+H]⁺: 194.1181; found: 194.1185.

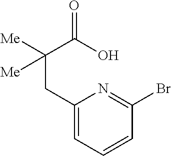 [SMILES: CC(C)(Cc1cccc(Br)n1)C(=O)O]

### 3-(6-Bromopyridin-2-yl)-2,2-dimethylpropanoic Acid (3h)

Following General Procedure. Purification by pTLC (30% EA/hexanes, Rf=0.35) afforded the title compound as colourless oil (14.1 mg, 0.06 mmol). ¹H NMR (600 MHz, CDCl₃) δ 7.50 (t, J=7.7 Hz, 1H), 7.38 (d, J=7.9 Hz, 1H), 7.17 (d, J=7.5 Hz, 1H), 3.08 (s, 2H), 1.28 (s, 6H). ¹³C NMR (151 MHz, CDCl₃) δ 181.23, 159.76, 140.85, 138.83, 126.09, 123.24, 46.51, 42.89, 25.39.

HRMS (ESI-TOF) Calcd for C₁₀H₁₃BrNO₂⁺ [M+H]⁺: 258.0130; found: 258.0126.

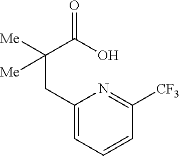 [SMILES: CC(C)(Cc1cccc(C(F)(F)F)n1)C(=O)O]

### 2,2-Dimethyl-3-(6-(trifluoromethyl)pyridin-2-yl)propanoic Acid (3i)

Following General Procedure. Purification by pTLC (30% EA/hexanes, Rf=0.30) afforded the title compound as colourless oil (16.3 mg, 0.07 mmol). ¹H NMR (600 MHz, CDCl₃) δ 7.81 (t, J=7.8 Hz, 1H), 7.56 (d, J=7.7 Hz, 1H), 7.39 (d, J=7.8 Hz, 1H), 3.19 (s, 2H), 1.29 (s, 6H). ¹³C NMR (151 MHz, CDCl₃) δ 182.38, 159.25, 147.28 (q, J_{CF}=39.8 Hz), 137.64, 127.03, 121.43 (q, J_{CF}=274.8 Hz), 118.19 (q, J_{CF}=5.6 Hz), 46.85, 42.73, 25.29. ¹⁹F NMR (376 MHz, CDCl₃) δ −70.81.

HRMS (ESI-TOF) Calcd for C₁₁H₁₃F₃NO₂⁺ [M+H]⁺: 248.0898; found: 248.0901.

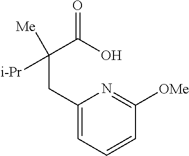 [SMILES: COc1cccc(CC(C)(C(=O)O)C(C)C)n1]

### 3-(6-Methoxypyridin-2-yl)-2,2-dimethylpropanoic Acid (3j)

Following General Procedure. Purification by pTLC (40% EA/hexanes, Rf=0.35) afforded the title compound as colourless oil (15.7 mg, 0.08 mmol). ¹H NMR (600 MHz, CDCl₃) δ 7.57 (t, J=7.8 Hz, 1H), 6.77 (d, J=7.1 Hz, 1H), 6.69 (d, J=8.3 Hz, 1H), 3.93 (s, 3H), 3.03 (s, 2H), 1.27 (s, 6H). ¹³C NMR (151 MHz, CDCl₃) δ 180.87, 163.20, 155.36, 139.85, 117.22, 109.41, 54.00, 45.70, 42.55, 26.16.

HRMS (ESI-TOF) Calcd for C₁₁H₁₆NO₃⁺ [M+H]⁺: 210.1130; found: 210.1132.

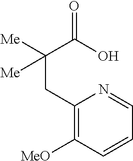 [SMILES: COc1cccnc1CC(C)(C)C(=O)O]

### 3-(3-Methoxypyridin-2-yl)-2,2-dimethylpropanoic Acid (3k)

Following General Procedure (Reaction time is modified to 48 hrs for this substrate). Purification by pTLC (40% EA/hexanes, Rf=0.25) afforded the title compound as colourless oil (7.3 mg, 0.04 mmol). ¹H NMR (600 MHz, CDCl₃) δ 8.12 (dd, J=4.0, 2.2 Hz, 1H), 7.31 (d, J=4.1 Hz, 2H), 3.91 (s, 3H), 3.16 (s, 2H), 1.28 (s, 6H). ¹³C NMR (151 MHz, CDCl₃) δ 179.18, 154.68, 148.03, 137.31, 123.15, 118.92, 55.65, 42.68, 36.64, 26.69.

HRMS (ESI-TOF) Calcd for C₁₁H₁₆NO₃⁺ [M+H]⁺: 210.1130; found: 210.1134.

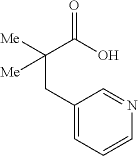 [SMILES: CC(C)(Cc1cccnc1)C(=O)O]

### 2,2-Dimethyl-3-(pyridin-3-yl)propanoic Acid (31)

Following General Procedure (Reaction time is modified to 48 hrs and concentration is 0.5 M for this substrate). Purification by pTLC (90% EA/hexanes, Rf=0.30) afforded the title compound as colourless oil (8.2 mg, 0.05 mmol). ¹H NMR (600 MHz, CDCl₃) δ 8.55 (s, 1H), 8.44 (d, J=4.6 Hz, 1H), 7.60 (d, J=7.7 Hz, 1H), 7.31 (dd, J=7.6, 5.0 Hz, 1H), 2.92 (s, 2H), 1.28 (s, 6H). ¹³C NMR (151 MHz, CDCl₃) δ 179.44, 148.76, 145.57, 139.37, 135.06, 123.42, 44.37, 44.00, 25.14.

HRMS (ESI-TOF) Calcd for C₁₀H₁₄NO₂⁺ [M+H]⁺: 180.1025; found: 180.1026.

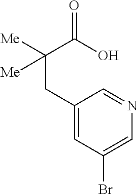 [SMILES: CC(C)(Cc1cncc(Br)c1)C(=O)O]

### 3-(5-Bromopyridin-3-yl)-2,2-dimethylpropanoic Acid (3m)

Following General Procedure (Reaction time is modified to 48 hrs and concentration is 0.5 M for this substrate). Purification by pTLC (35% EA/hexanes, Rf=0.30) afforded the title compound as colourless oil (8.5 mg, 0.03 mmol). ¹H NMR (600 MHz, CDCl₃) δ 8.55 (s, 1H), 8.43 (s, 1H), 7.73 (s, 1H), 2.89 (s, 2H), 1.28 (s, 6H). ¹³C NMR (151 MHz, CDCl₃) δ 179.15, 148.02, 147.90, 141.17, 136.08, 120.34, 43.65, 43.37, 25.00. HRMS (ESI-TOF) Calcd for C₁₀H₁₃BrNO₂⁺ [M+H]⁺: 258.0130; found: 258.0131.

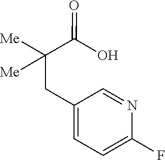 [SMILES: CC(C)(Cc1ccc(F)nc1)C(=O)O]

### 3-(6-Fluoropyridin-3-yl)-2,2-dimethylpropanoic Acid (3n)

Following General Procedure (Reaction time is modified to 48 hrs for this substrate). Purification by pTLC (30% EA/hexanes, Rf=0.35) afforded the title compound as colourless oil (12.6 mg, 0.06 mmol) (Notice: This is a mixture of mono- and di-heteroarylated products). ¹H NMR (600 MHz, CDCl₃) δ 7.99 (dd, J=7.6, 2.3 Hz, 1.5H), 7.57 (dd, J=8.5, 2.4 Hz, 1.5H), 6.91 (t, J=8.3 Hz, 1.5H), 6.60-6.50 (m, 1.5H), 3.18 (d, J=13.7 Hz, 0.5H), 2.87 (s, 2H), 2.73 (s, 0.5H), 1.25 (s, 6H). ¹³C NMR (151 MHz, CDCl₃) δ 182.91, 158.98, 147.13, 141.89, 129.04, 110.43, 43.30, 41.88, 24.69. ¹⁹F NMR (376 MHz, CDCl₃) δ −76.01.

HRMS (ESI-TOF) Calcd for C₁₀H₁₃FNO₂⁺ [M−H]⁻: 196.0774; found: 196.0776.

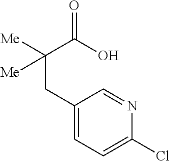 [SMILES: CC(C)(Cc1ccc(Cl)nc1)C(=O)O]

### 3-(6-Chloropyridin-3-yl)-2,2-dimethylpropanoic Acid (30)

Following General Procedure. Purification by pTLC (30% EA/hexanes, Rf=0.35) afforded the title compound as colourless oil (13.2 mg, 0.06 mmol). ¹H NMR (600 MHz, CDCl₃) δ 8.28 (s, 1H), 7.52 (d, J=8.2 Hz, 1H), 7.29 (d, J=8.2 Hz, 1H), 2.89 (s, 2H), 1.26 (s, 6H). ¹³C NMR (151 MHz, CDCl₃) δ 181.58, 150.60, 149.57, 140.78, 132.49, 123.83, 43.40, 42.36, 24.85.

HRMS (ESI-TOF) Calcd for C₁₀H₁₃ClNO₂⁺ [M+H]⁺: 214.0635; found: 214.0638.

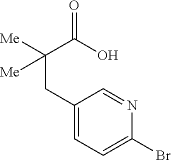 [SMILES: CC(C)(Cc1ccc(Br)nc1)C(=O)O]

### 3-(6-Bromopyridin-3-yl)-2,2-dimethylpropanoic Acid (3p)

Following General Procedure. Purification by pTLC (30% EA/hexanes, Rf=0.35) afforded the title compound as colourless oil (13.6 mg, 0.05 mmol). ¹H NMR (600 MHz, CDCl₃) δ 8.25 (s, 1H), 7.47-7.39 (m, 2H), 2.87 (s, 2H), 1.25 (s, 6H). ¹³C NMR (151 MHz, CDCl₃) δ 181.18, 151.28, 140.49, 140.11, 132.80, 127.60, 43.29, 42.37, 24.87.

HRMS (ESI-TOF) Calcd for C₁₀H₁₃BrNO₂⁺ [M+H]⁺: 258.0130; found: 258.0126.

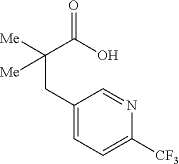 [SMILES: CC(C)(Cc1ccc(C(F)(F)F)nc1)C(=O)O]

### 2,2-Dimethyl-3-(6-(trifluoromethyl)pyridin-3-yl)propanoic Acid (3q)

Following General Procedure. Purification by pTLC (30% EA/hexanes, Rf=0.30) afforded the title compound as colourless oil (17.3 mg, 0.07 mmol). ¹H NMR (600 MHz, CDCl₃) δ 8.62 (s, 1H), 7.73 (d, J=9.7 Hz, 1H), 7.65 (d, J=8.0 Hz, 1H), 3.00 (s, 2H), 1.28 (s, 6H). ¹³C NMR (151 MHz, CDCl₃) δ 181.74, 151.20, 146.39 (q, J_{CF}=39.8 Hz), 139.09, 136.93, 121.59 (q, J_{CF}=548.1 Hz), 120.03 (q, J_{CF}=5.3 Hz), 43.40, 42.77, 24.92. ¹⁹F NMR (376 MHz, CDCl₃) δ −70.33.

HRMS (ESI-TOF) Calcd for C₁₁H₁₃F₃NO₂⁺ [M+H]⁺: 248.0898; found: 248.0898.

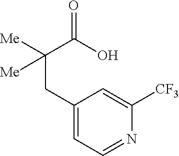 [SMILES: CC(C)(Cc1ccnc(C(F)(F)F)c1)C(=O)O]

### 2,2-Dimethyl-3-(2-(trifluoromethyl)pyridin-4-yl)propanoic Acid (3r)

Following General Procedure. Purification by pTLC (45% EA/hexanes, Rf=0.35) afforded the title compound as colourless oil (16.8 mg, 0.07 mmol). ¹H NMR (600 MHz, CDCl₃) δ 8.67 (d, J=4.9 Hz, 1H), 7.54 (s, 1H), 7.35 (d, J=4.8 Hz, 1H), 3.00 (s, 2H), 1.28 (s, 6H). ¹³C NMR (151 MHz, CDCl₃) δ 182.14, 149.67, 148.93, 148.02 (q, J_{CF}=39.7 Hz), 128.18, 121.54 (q, J_{CF}=460.6 Hz), 122.19 (q, J_{CF}=6.0 Hz), 45.04, 43.25, 24.91. ¹⁹F NMR (376 MHz, CDCl₃) δ −70.60.

HRMS (ESI-TOF) Calcd for C₁₁H₁₃F₃NO₂⁺ [M+H]⁺: 248.0898; found: 248.0900.

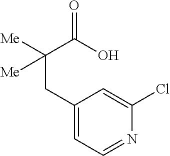 [SMILES: CC(C)(Cc1ccnc(Cl)c1)C(=O)O]

### 3-(2-Chloropyridin-4-yl)-2,2-dimethylpropanoic Acid (3s)

Following General Procedure. Purification by pTLC (30% EA/hexanes, Rf=0.35) afforded the title compound as colourless oil (11.9 mg, 0.06 mmol). ¹H NMR (600 MHz, CDCl₃) δ 8.32 (d, J=4.9 Hz, 1H), 7.19 (s, 1H), 7.08 (d, J=4.6 Hz, 1H), 2.90 (s, 2H), 1.27 (s, 6H). ¹³C NMR (151 MHz, CDCl₃) δ 181.64, 151.42, 150.24, 149.23, 125.88, 124.33, 44.71, 43.13, 24.98.

HRMS (ESI-TOF) Calcd for C₁₀H₁₃ClNO₂⁺ [M+H]⁺: 214.0635; found: 214.0633.

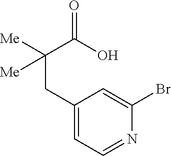 [SMILES: CC(C)(Cc1ccnc(Br)c1)C(=O)O]

### 3-(2-Bromopyridin-4-yl)-2,2-dimethylpropanoic Acid (3t)

Following General Procedure. Purification by pTLC (30% EA/hexanes, Rf=0.35) afforded the title compound as colourless oil (11.1 mg, 0.04 mmol). ¹H NMR (600 MHz, CDCl₃) δ 8.30 (d, J=5.0 Hz, 1H), 7.35 (s, 1H), 7.11 (d, J=4.2 Hz, 1H), 2.88 (s, 2H), 1.27 (s, 6H). ¹³C NMR (151 MHz, CDCl₃) δ 181.29, 149.95, 149.70, 142.17, 129.67, 124.69, 44.61, 43.11, 24.98.

HRMS (ESI-TOF) Calcd for C₁₀H₁₃BrNO₂⁺ [M+H]⁺: 258.0130; found: 258.0131.

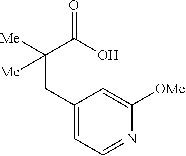 [SMILES: COc1cc(CC(C)(C)C(=O)O)ccn1]

### 3-(2-Methoxypyridin-4-yl)-2,2-dimethylpropanoic Acid (3u)

Following General Procedure. Purification by pTLC (30% EA/hexanes, Rf=0.30) afforded the title compound as colourless oil (10.5 mg, 0.05 mmol). ¹H NMR (600 MHz, CDCl₃) δ 8.09 (d, J=5.2 Hz, 1H), 6.73 (d, J=5.2 Hz, 1H), 6.59 (s, 1H), 3.94 (s, 3H), 2.86 (s, 2H), 1.25 (s, 6H). ¹³C NMR (151 MHz, CDCl₃) δ 182.35, 164.28, 149.50, 146.33, 119.00, 112.22, 53.42, 44.98, 43.03, 24.91.

HRMS (ESI-TOF) Calcd for C₁₁H₁₆NO₃⁺ [M+H]⁺: 210.1130; found: 210.1132.

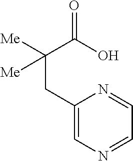 [SMILES: CC(C)(Cc1cnccn1)C(=O)O]

### 2,2-Dimethyl-3-(pyrazin-2-yl)propanoic Acid (3v)

Following General Procedure. Purification by pTLC (50% EA/hexanes, Rf=0.25) afforded the title compound as colourless oil (9.6 mg, 0.05 mmol). ¹H NMR (600 MHz, CDCl₃) δ 8.58 (s, 1H), 8.54 (s, 1H), 8.48 (s, 1H), 3.15 (s, 2H), 1.31 (s, 6H). ¹³C NMR (151 MHz, CDCl₃) δ 180.84, 154.42, 145.40, 143.71, 142.31, 44.83, 43.04, 25.27. δ 166.48, 145.76, 145.54, 143.84, 143.13, 142.92, 136.22, 130.20, 130.16, 128.12, 121.18, 60.86, 14.33.

HRMS (ESI-TOF) Calcd for C₁₀H₁₁N₂O₂⁺ [M−H]⁻: 179.0821; found: 179.0824.

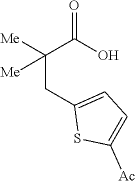 [SMILES: CC(=O)c1ccc(CC(C)(C)C(=O)O)s1]

### 3-(5-Acetylthiophen-2-yl)-2,2-dimethylpropanoic Acid (3w)

Following General Procedure. Purification by pTLC (25% EA/hexanes, Rf=0.40) afforded the title compound as colourless oil (9.9 mg, 0.04 mmol). ¹H NMR (600 MHz, CDCl₃) δ 7.58 (d, J=3.7 Hz, 1H), 6.88 (d, J=3.7 Hz, 1H), 3.14 (s, 2H), 2.54 (s, 3H), 1.30 (s, 6H). ¹³C NMR (151 MHz, CDCl₃) δ 190.65, 182.29, 149.35, 143.31, 132.66, 128.45, 43.50, 40.54, 26.56, 24.90.

HRMS (ESI-TOF) Calcd for C₁₁H₁₅O₃S⁺ [M+H]⁺: 227.0742; found: 227.0742.

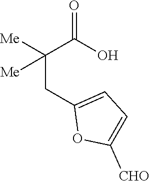 [SMILES: CC(C)(Cc1ccc(C=O)o1)C(=O)O]

### 3-(5-Formylfuran-2-yl)-2,2-dimethylpropanoic Acid (3x)

Following General Procedure (Reaction concentration is modified to 0.5 M for this substrate). Purification by pTLC (25% EA/hexanes, Rf=0.35) afforded the title compound as colourless oil (9.6 mg, 0.04 mmol). ¹H NMR (600 MHz, CDCl₃) δ 9.57 (s, 1 H), 7.21 (d, J=3.4 Hz, 1H), 6.35 (d, J=3.4 Hz, 1H), 3.05 (s, 2H), 1.31 (s, 6H). ¹³C NMR (151 MHz, CDCl₃) δ 181.12, 177.19, 159.81, 152.20, 111.29, 42.80, 38.33, 24.97. HRMS (ESI-TOF) Calcd for C₁₀H₁₃O₄⁺ [M+H]⁺: 197.0814; found: 197.0810.

### Scope of Carboxylic Acids for β-C(Sp³)-H Heteroarylation

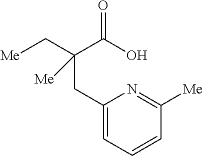 [SMILES: CCC(C)(Cc1cccc(C)n1)C(=O)O]

### 2-Methyl-2-((6-methylpyridin-2-yl)methyl)butanoic Acid (4a)

Following General Procedure. Purification by pTLC (90% EA/hexanes, Rf=0.30) afforded the title compound as colourless oil (15.9 mg, 0.08 mmol). ¹H NMR (600 MHz, CDCl₃) δ 7.70 (t, J=8.0 Hz, 1H), 7.17 (d, J=7.4 Hz, 1H), 7.11 (d, J=7.4 Hz, 1H), 3.13-2.98 (m, 2H), 2.61 (s, 3H), 1.75-1.64 (m, 2H), 1.21 (s, 3H), 0.92 (t, J=7.4 Hz, 3H). ¹³C NMR (151 MHz, CDCl₃) δ 178.43, 156.87, 155.90, 139.11, 122.60, 122.25, 46.79, 42.62, 32.17, 24.60, 22.81, 8.84.

HRMS (ESI-TOF) Calcd for C₁₂H₁₈NO₂⁺ [M+H]⁺: 208.1338; found: 208.1344.

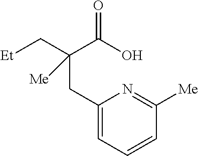 [SMILES: CCCC(C)(Cc1cccc(C)n1)C(=O)O]

### 2-Methyl-2-((6-methylpyridin-2-yl)methyl)pentanoic Acid (4b)

Following General Procedure. Purification by pTLC (90% EA/hexanes, Rf=0.35) afforded the title compound as colourless oil (19.0 mg, 0.09 mmol). ¹H NMR (600 MHz, CDCl₃) δ 7.70 (t, J=7.7 Hz, 1H), 7.17 (d, J=7.8 Hz, 1H), 7.10 (d, J=7.7 Hz, 1H), 3.10-3.01 (m, 2H), 2.61 (s, 3H), 1.63-1.56 (m, 1H), 1.47-1.40 (m, 1H), 1.39-1.32 (m, 2H), 1.20 (s, 3H), 0.85 (t, J=7.2 Hz, 3H). ¹³C NMR (151 MHz, CDCl₃) δ 178.58, 156.88, 155.89, 139.10, 122.57, 122.25, 46.59, 43.04, 41.89, 25.06, 22.83, 17.66, 14.53.

HRMS (ESI-TOF) Calcd for C₁₃H₂₀NO₂⁺ [M+H]⁺: 222.1494; found: 222.1491.

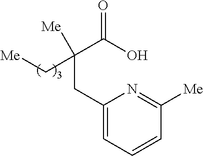

### 2-Methyl-2-((6-methylpyridin-2-yl)methyl)hexanoic Acid (4c)

Following General Procedure. Purification by pTLC (90% EA/hexanes, Rf=0.30) afforded the title compound as colourless oil (17.4 mg, 0.07 mmol). ¹H NMR (600 MHz, CDCl₃) δ 7.70 (t, J=7.7 Hz, 1H), 7.17 (d, J=7.7 Hz, 1H), 7.10 (d, J=7.6 Hz, 1H), 3.10-3.00 (m, 2H), 2.61 (s, 3H), 1.61 (ddd, J=13.5, 10.1, 6.3 Hz, 1H), 1.49-1.43 (m, 1H), 1.33-1.28 (m, 2H), 1.24 (ddd, J=7.2, 5.5, 1.1 Hz, 2H), 1.20 (s, 3H), 0.84 (t, J=7.2 Hz, 3H). ¹³C NMR (151 MHz, CDCl₃) δ 178.64, 156.88, 155.87, 139.10, 122.59, 122.25, 46.50, 42.96, 39.24, 26.51, 25.04, 23.11, 22.82, 13.91.

HRMS (ESI-TOF) Calcd for C₁₇H₂₈NO₂⁺ [M+H]⁺: 236.1651; found: 236.1659.

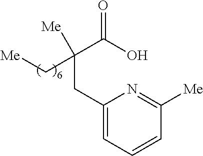

### 2-Methyl-2-((6-methylpyridin-2-yl)methyl)nonanoic Acid (4d)

Following General Procedure. Purification by pTLC (90% EA/hexanes, Rf=0.35) afforded the title compound as colourless oil (18.0 mg, 0.07 mmol). ¹H NMR (600 MHz, CDCl₃) δ 7.69 (t, J=7.7 Hz, 1H), 7.17 (d, J=7.8 Hz, 1H), 7.10 (d, J=7.5 Hz, 1H), 3.10-2.99 (m, 2H), 2.60 (s, 3H), 1.60 (ddd, J=13.4, 10.3, 6.3 Hz, 1H), 1.48-1.41 (m, 1H), 1.33-1.18 (m, 13H), 0.87 (t, J=7.1 Hz, 3H). ¹³C NMR (151 MHz, CDCl₃) δ 178.79, 156.87, 155.86, 139.09, 122.59, 122.25, 46.55, 43.00, 39.49, 31.74, 29.98, 29.08, 24.98, 24.30, 22.81, 22.62, 14.07.

HRMS (ESI-TOF) Calcd for C₁₃H₁₃N₂O₂⁺ [M+H]⁺: 278.2120; found: 278.2126.

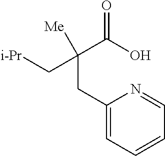 [SMILES: CC(C)CC(C)(Cc1ccccn1)C(=O)O]

### 2,4-Dimethyl-2-(pyridin-2-ylmethyl)pentanoic Acid (4e)

Following General Procedure. Purification by pTLC (40% EA/hexanes, Rf=0.35) afforded the title compound as colourless oil (13.2 mg, 0.06 mmol). ¹H NMR (600 MHz, CDCl₃) δ 8.56 (d, J=4.5 Hz, 1H), 7.76 (td, J=7.7, 1.7 Hz, 1H), 7.30 (dd, J=7.3, 5.6 Hz, 1H), 7.27 (d, J=7.8 Hz, 1H), 3.20 (d, J=14.8 Hz, 1H), 3.02 (d, J=14.8 Hz, 1H), 1.81 (qd, J=6.8, 5.7 Hz, 1H), 1.63-1.52 (m, 2H), 1.21 (s, 3H), 0.93 (d, J=6.6 Hz, 3H), 0.89 (d, J=6.7 Hz, 3H). ¹³C NMR (151 MHz, CDCl₃) δ 179.68, 157.61, 146.91, 138.19, 125.54, 122.29, 48.27, 45.32, 24.75, 24.46, 23.85, 23.63.

HRMS (ESI-TOF) Calcd for C₁₃H₂₀NO₂⁺ [M+H]⁺: 222.1494; found: 222.1494.

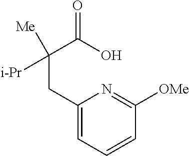 [SMILES: COc1cccc(CC(C)(C(=O)O)C(C)C)n1]

### 2-((6-Methoxypyridin-2-yl)methyl)-2,3-dimethylbutanoic Acid (4f)

Following General Procedure. Purification by pTLC (40% EA/hexanes, Rf=0.30) afforded the title compound as colourless oil (14.7 mg, 0.06 mmol). ¹H NMR (600 MHz, CDCl₃) δ 7.60 (dd, J=8.4, 7.2 Hz, 1H), 6.82 (d, J=7.2 Hz, 1H), 6.71 (d, J=8.4 Hz, 1H), 3.96 (s, 3H), 3.04-2.96 (m, 2H), 2.12 (p, J=6.8 Hz, 1H), 1.12 (s, 3H), 0.96 (d, J=6.8 Hz, 3H), 0.88 (d, J=6.8 Hz, 3H). ¹³C NMR (151 MHz, CDCl₃) δ 178.97, 163.01, 155.30, 140.27, 117.72, 109.67, 54.35, 49.79, 41.48, 32.94, 19.19, 17.60, 17.35.

HRMS (ESI-TOF) Calcd for C₁₃H₂₀NO₃⁺ [M+H]⁺: 238.1443; found: 238.1446.

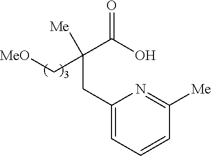

### 5-Methoxy-2-methyl-2-((6-methylpyridin-2-yl)methyl)pentanoic Acid (4g)

Following General Procedure. Purification by pTLC (90% EA/hexanes, Rf=0.30) afforded the title compound as colourless oil (15.6 mg, 0.06 mmol). ¹H NMR (600 MHz, CDCl₃) δ 7.70 (t, J=7.7 Hz, 1H), 7.18 (d, J=7.8 Hz, 1H), 7.10 (d, J=7.9 Hz, 1H), 3.34 (t, J=6.3 Hz, 2H), 3.30 (s, 3H), 3.14 (d, J=15.5 Hz, 1H), 2.99 (d, J=15.5 Hz, 1H), 2.61 (s, 3H), 1.76-1.70 (m, 1H), 1.68-1.59 (m, 2H), 1.54 (ddd, J=13.0, 11.8, 4.6 Hz, 1H), 1.19 (s, 3H). ¹³C NMR (151 MHz, CDCl₃) δ 178.38, 156.67, 155.88, 139.21, 122.65, 122.34, 72.80, 58.44, 46.23, 42.83, 36.49, 24.93, 24.66, 22.79.

HRMS (ESI-TOF) Calcd for C₁₄H₂₂NO₃⁺ [M+H]⁺: 252.1600; found: 252.1600.

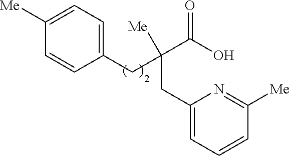

### 2-Methyl-2-((6-methylpyridin-2-yl)methyl)-4-(p-tolyl) butanoic Acid (4h)

Following General Procedure. Purification by pTLC (90% EA/hexanes, Rf=0.30) afforded the title compound as colourless oil (24.1 mg, 0.08 mmol). ¹H NMR (600 MHz, CDCl₃) δ 7.70 (t, J=7.7 Hz, 1H), 7.18 (d, J=7.8 Hz, 1H), 7.13 (d, J=7.6 Hz, 1H), 7.06 (d, J=7.7 Hz, 2H), 7.00 (d, J=8.0 Hz, 2H), 3.19-3.08 (m, 2H), 2.66 (td, J=12.9, 4.7 Hz, 1H), 2.61 (s, 3H), 2.57 (td, J=12.9, 4.9 Hz, 1H), 2.31 (s, 3H), 1.90 (ddd, J=13.6, 12.7, 4.6 Hz, 1H), 1.73 (ddd, J=13.6, 12.6, 5.0 Hz, 1H), 1.30 (s, 3H). ¹³C NMR (151 MHz, CDCl₃) δ 178.14, 156.54, 155.97, 139.22, 139.01, 135.32, 129.06, 128.24, 122.65, 122.41, 46.59, 43.12, 41.92, 30.52, 25.15, 22.81, 20.97.

HRMS (ESI-TOF) Calcd for C₁₉H₂₄NO₂⁺ [M+H]⁺: 298.1807; found: 298.1820.

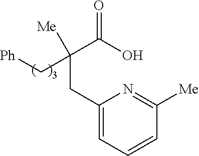

### 2-Methyl-2-((6-methylpyridin-2-yl)methyl)-5-phenylpentanoic Acid (4i)

Following General Procedure. Purification by pTLC (90% EA/hexanes, Rf=0.30) afforded the title compound as colourless oil (20.8 mg, 0.07 mmol). ¹H NMR (600 MHz, CDCl₃) δ 7.68 (t, J=7.7 Hz, 1H), 7.25 (t, J=7.4 Hz, 2H), 7.17 (dd, J=14.8, 7.5 Hz, 2H), 7.10 (d, J=6.9 Hz, 2H), 7.07 (d, J=7.4 Hz, 1H), 3.09-2.95 (m, 2H), 2.57 (d, J=0.6 Hz, 3H), 2.54 (t, J=7.2 Hz, 2H), 1.71-1.64 (m, 3H), 1.53-1.47 (m, 1H), 1.20 (s, 3H). ¹³C NMR (151 MHz, CDCl₃) δ 178.46, 156.65, 155.85, 142.03, 139.11, 128.36, 128.23, 125.71, 122.53, 122.29, 46.40, 42.71, 38.93, 36.09, 26.12, 25.00, 22.76.

HRMS (ESI-TOF) Calcd for C₁₉H₂₄NO₂⁺ [M+H]⁺: 298.1807; found: 298.1807.

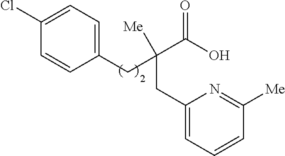

### 4-(4-Chlorophenyl)-2-methyl-2-((6-methylpyridin-2-yl)methyl)butanoic Acid (4j)

Following General Procedure. Purification by pTLC (90% EA/hexanes, Rf=0.30) afforded the title compound as colourless oil (21.2 mg, 0.07 mmol). ¹H NMR (600 MHz, CDCl₃) δ 7.71 (t, J=7.7 Hz, 1H), 7.24-7.17 (m, 3H), 7.13 (d, J=7.6 Hz, 1H), 7.06 (d, J=8.4 Hz, 2H), 3.20 (d, J=15.5 Hz, 1H), 3.07 (d, J=15.5 Hz, 1H), 2.68 (td, J=13.0, 4.5 Hz, 1H), 2.62-2.56 (m, 4H), 1.97-1.90 (m, 1H), 1.70 (ddd, J=13.6, 12.5, 5.0 Hz, 1H), 1.26 (s, 3H). ¹³C NMR (151 MHz, CDCl₃) δ 178.00, 156.40, 156.03, 140.56, 139.30, 131.56, 129.71, 128.46, 122.64, 122.50, 46.57, 43.01, 42.07, 30.45, 25.03, 22.81.

HRMS (ESI-TOF) Calcd for C₁₈H₂₁ClNO₂⁺ [M+H]⁺: 318.1261; found: 318.1261.

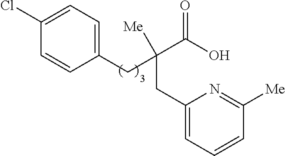

### 5-(4-Chlorophenyl)-2-methyl-2-((6-methylpyridin-2-yl)methyl)pentanoic Acid (4k)

Following General Procedure. Purification by pTLC (90% EA/hexanes, Rf=0.30) afforded the title compound as colourless oil (20.5 mg, 0.06 mmol). ¹H NMR (600 MHz, CDCl₃) δ 7.67 (t, J=7.7 Hz, 1H), 7.19 (d, J=8.4 Hz, 2H), 7.16 (d, J=7.8 Hz, 1H), 7.05 (d, J=7.7 Hz, 1H), 7.01 (d, J=8.4 Hz, 2H), 3.07-2.95 (m, 2H), 2.56 (s, 3H), 2.54-2.49 (m, 2H), 1.68-1.60 (m, 3H), 1.47-1.40 (m, 1H), 1.20 (s, 3H). ¹³C NMR (151 MHz, CDCl₃) δ 178.42, 156.50, 155.87, 140.40, 139.15, 131.39, 129.73, 128.29, 122.51, 122.30, 46.33, 42.71, 38.62, 35.31, 25.95, 24.98, 22.71.

HRMS (ESI-TOF) Calcd for C₁₉H₂₃ClNO₂⁺ [M+H]⁺: 332.1417; found: 332.1416.

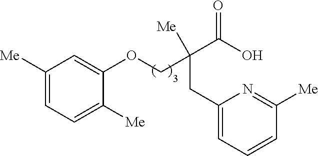

### 5-(2,5-Dimethylphenoxy)-2-methyl-2-((6-methylpyridin-2-yl)methyl)pentanoic Acid (4l)

Following General Procedure. Purification by pTLC (90% EA/hexanes, Rf=0.35) afforded the title compound as colourless oil (21.1 mg, 0.06 mmol). ¹H NMR (600 MHz, CDCl₃) δ 7.69 (t, J=7.7 Hz, 1H), 7.17 (d, J=7.8 Hz, 1H), 7.11 (d, J=7.6 Hz, 1H), 6.99 (d, J=7.5 Hz, 1H), 6.66 (d, J=7.5 Hz, 1H), 6.58 (s, 1H), 3.89 (dtd, J=15.4, 9.1, 6.2 Hz, 2H), 3.11 (s, 2H), 2.61 (s, 3H), 2.31 (s, 3H), 2.10 (s, 3H), 1.92-1.73 (m, 3H), 1.69 (ddd, J=13.1, 11.6, 4.8 Hz, 1H), 1.28 (s, 3H). ¹³C NMR (151 MHz, CDCl₃) δ 178.24, 156.83, 156.54, 155.92, 139.25, 136.52, 130.22, 123.41, 122.64, 122.39, 120.66, 111.87, 67.71, 46.22, 43.06, 36.03, 25.11, 24.63, 22.78, 21.39, 15.74.

HRMS (ESI-TOF) Calcd for C₂₁H₂₈NO₃⁺ [M+H]⁺: 342.2069; found: 342.2075.

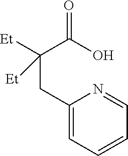 [SMILES: CCC(CC)(Cc1ccccn1)C(=O)O]

### 2-Ethyl-2-(pyridin-2-ylmethyl)butanoic Acid (4m)

Following General Procedure. Purification by pTLC (35% EA/hexanes, Rf=0.30) afforded the title compound as colourless oil (8.3 mg, 0.04 mmol). ¹H NMR (600 MHz, CDCl₃) δ 8.53 (d, J=4.6 Hz, 1H), 7.80 (td, J=7.7, 1.8 Hz, 1H), 7.33 (ddd, J=8.8, 6.9, 2.5 Hz, 2H), 3.11 (s, 2H), 1.69 (dq, J=14.8, 7.5 Hz, 2H), 1.52 (dq, J=14.6, 7.4 Hz, 2H), 0.91 (t, J=7.4 Hz, 6H). ¹³C NMR (151 MHz, CDCl₃) δ 177.20, 158.03, 146.38, 138.68, 125.71, 122.31, 50.42, 40.86, 29.41, 8.63.

HRMS (ESI-TOF) Calcd for C₁₂H₁₈NO₂⁺ [M+H]⁺: 208.1338; found: 208.1336.

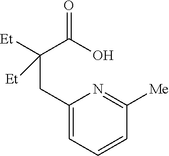 [SMILES: CCC(CC)(Cc1cccc(C)n1)C(=O)O]

### 2-Ethyl-2-((6-methylpyridin-2-yl)methyl)butanoic Acid (4n)

Following General Procedure. Purification by pTLC (90% EA/hexanes, Rf=0.35) afforded the title compound as colourless oil (9.1 mg, 0.04 mmol). ¹H NMR (600 MHz, CDCl₃) δ 7.70 (t, J=7.7 Hz, 1H), 7.17 (d, J=7.5 Hz, 1H), 7.12 (d, J=7.7 Hz, 1H), 3.06 (s, 2H), 2.61 (s, 3H), 1.70 (dq, J=13.7, 7.4 Hz, 2H), 1.48 (dq, J=13.8, 7.4 Hz, 2H), 0.90 (t, J=7.4 Hz, 6H). ¹³C NMR (151 MHz, CDCl₃) δ 177.45, 157.13, 155.85, 139.12, 122.74, 122.20, 50.51, 40.48, 29.93, 22.79, 8.69.

HRMS (ESI-TOF) Calcd for C₁₃H₂₀NO₂⁺ [M+H]⁺: 222.1494; found: 222.1502.

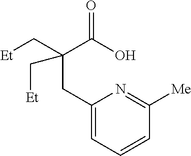 [SMILES: CCCC(CCC)(Cc1cccc(C)n1)C(=O)O]

### 2-((6-Methylpyridin-2-yl)methyl)-2-propylpentanoic Acid (4o)

Following General Procedure. Purification by pTLC (90% EA/hexanes, Rf=0.35) afforded the title compound as colourless oil (11.5 mg, 0.05 mmol). ¹H NMR (600 MHz, CDCl₃) δ 7.70 (t, J=7.7 Hz, 1H), 7.17 (d, J=7.8 Hz, 1H), 7.11 (d, J=7.6 Hz, 1H), 3.07 (s, 2H), 2.61 (s, 3H), 1.60 (td, J=12.2, 11.5, 3.3 Hz, 2H), 1.41-1.28 (m, 6H), 0.85 (t, J=7.1 Hz, 6H). ¹³C NMR (151 MHz, CDCl₃) δ 177.72, 157.13, 155.82, 139.12, 122.68, 122.21, 50.26, 41.20, 40.12, 22.81, 17.50, 14.62.

HRMS (ESI-TOF) Calcd for C₁₅H₂₄NO₂⁺ [M+H]⁺: 250.1807; found: 250.1814.

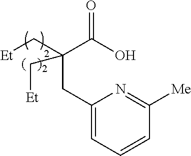

### 2-Butyl-2-((6-methylpyridin-2-yl)methyl) hexanoic Acid (4p)

Following General Procedure. Purification by pTLC (90% EA/hexanes, Rf=0.35) afforded the title compound as colourless oil (15.2 mg, 0.06 mmol). ¹H NMR (600 MHz, CDCl₃) δ 7.69 (t, J=7.7 Hz, 1H), 7.17 (d, J=7.7 Hz, 1H), 7.11 (d, J=7.6 Hz, 1H), 3.07 (s, 2H), 2.60 (s, 3H), 1.65-1.58 (m, 2H), 1.42 (ddd, J=13.5, 11.8, 4.5 Hz, 2H), 1.34-1.19 (m, 8H), 0.85 (t, J=7.1 Hz, 6H). ¹³C NMR (151 MHz, CDCl₃) δ 177.82, 157.16, 155.80, 139.08, 122.70, 122.18, 50.08, 41.12, 37.48, 26.38, 23.24, 22.80, 13.96.

HRMS (ESI-TOF) Calcd for C₁₇H₂₈NO₂⁺[M+H]⁺: 278.2120; found: 278.2122.

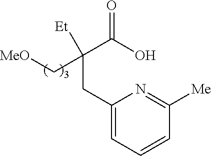

### 2-Ethyl-5-methoxy-2-((6-methylpyridin-2-yl)methyl)pentanoic Acid (4q)

Following General Procedure. Purification by pTLC (90% EA/hexanes, Rf=0.30) afforded the title compound as colourless oil (10.9 mg, 0.04 mmol). ¹H NMR (600 MHz, CDCl₃) δ 7.70 (t, J=7.7 Hz, 1H), 7.17 (d, J=7.8 Hz, 1H), 7.12 (d, J=7.6 Hz, 1H), 3.35 (td, J=6.3, 2.7 Hz, 2H), 3.31 (d, J=1.8 Hz, 3H), 3.14 (d, J=15.4 Hz, 1H), 3.04 (d, J=12.6 Hz, 1H), 2.61 (s, 3H), 1.76 (td, J=12.7, 4.1 Hz, 1H), 1.63 (dddd, J=18.8, 17.2, 9.6, 5.5 Hz, 3H), 1.53-1.46 (m, 2H), 0.89 (t, J=7.4 Hz, 3H). ¹³C NMR (151 MHz, CDCl₃) δ 177.35, 156.89, 155.83, 139.20, 122.78, 122.27, 72.99, 58.48, 50.00, 40.78, 34.04, 30.19, 24.49, 22.78, 8.67.

HRMS (ESI-TOF) Calcd for C₁₅H₂₄NO₃⁺ [M+H]⁺: 266.1756; found: 266.1761.

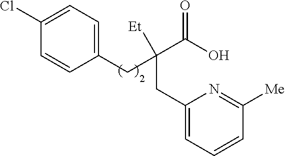

### 4-(4-Chlorophenyl)-2-ethyl-2-((6-methylpyridin-2-yl)methyl)butanoic Acid (4r)

Following General Procedure. Purification by pTLC (90% EA/hexanes, Rf=0.35) afforded the title compound as colourless oil (13.2 mg, 0.04 mmol). ¹H NMR (600 MHz, CDCl₃) δ 7.72 (t, J=7.7 Hz, 1H), 7.26-7.21 (m, 2H), 7.19 (d, J=7.8 Hz, 1H), 7.15 (d, J=7.6 Hz, 1H), 7.11-7.05 (m, 2H), 3.20 (d, J=15.4 Hz, 1H), 3.10 (d, J=15.4 Hz, 1H), 2.69 (td, J=13.0, 4.4 Hz, 1H), 2.62 (s, 3H), 2.56 (td, J=13.0, 5.0 Hz, 1H), 1.97 (td, J=13.1, 4.3 Hz, 1H), 1.72 (dt, J=14.8, 7.2 Hz, 1H), 1.68-1.62 (m, 1H), 1.56 (dq, J=13.8, 7.4 Hz, 1H), 0.92 (t, J=7.4 Hz, 3H). ¹³C NMR (151 MHz, CDCl₃) δ 177.04, 156.64, 156.00, 140.81, 139.27, 131.54, 129.76, 128.45, 122.76, 122.44, 50.33, 40.86, 39.86, 30.33, 30.29, 22.81, 8.66.

HRMS (ESI-TOF) Calcd for C₁₉H₂₃ClNO₂⁺ [M+H]⁺: 332.1417; found: 332.1432.

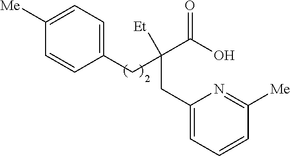

### 2-Ethyl-2-((6-methylpyridin-2-yl)methyl)-4-(p-tolyl)butanoic Acid (4s)

Following General Procedure. Purification by pTLC (90% EA/hexanes, Rf=0.35) afforded the title compound as colourless oil (12.4 mg, 0.04 mmol). ¹H NMR (600 MHz, CDCl₃) δ 7.70 (t, J=7.7 Hz, 1H), 7.16 (dd, J=16.2, 7.7 Hz, 2H), 7.07 (d, J=7.8 Hz, 2H), 7.03 (d, J=8.0 Hz, 2H), 3.15 (d, J=2.1 Hz, 2H), 2.66 (td, J=13.0, 4.4 Hz, 1H), 2.61 (s, 3H), 2.54 (td, J=13.0, 4.9 Hz, 1H), 2.31 (s, 3H), 1.94 (td, J=13.2, 4.4 Hz, 1H), 1.78 (dd, J=14.1, 7.3 Hz, 1H), 1.67 (ddd, J=13.6, 12.7, 4.8 Hz, 1H), 1.59 (dt, J=13.9, 7.3 Hz, 1H), 0.94 (t, J=7.4 Hz, 3H). ¹³C NMR (151 MHz, CDCl₃) δ 177.26, 156.83, 155.95, 139.25, 139.18, 135.28, 129.05, 128.29, 122.76, 122.33, 50.35, 40.89, 39.89, 30.42, 30.36, 22.82, 20.98, 8.72.

HRMS (ESI-TOF) Calcd for C₂₀H₂₆NO₂⁺ [M+H]⁺: 312.1964; found: 312.1966.

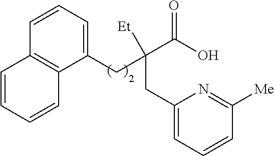

### 2-Ethyl-2-((6-methylpyridin-2-yl)methyl)-4-(naphthalen-1-yl)butanoic Acid (4t)

Following General Procedure. Purification by pTLC (90% EA/hexanes, Rf=0.35) afforded the title compound as colourless oil (17.4 mg, 0.05 mmol). ¹H NMR (600 MHz, CDCl₃) δ 8.07 (s, 1H), 7.84 (s, 1H), 7.78-7.67 (m, 2H), 7.50 (t, J=15.9 Hz, 2H), 7.39 (d, J=17.4 Hz, 1H), 7.32-7.25 (m, 2H), 7.23-7.14 (m, 2H), 3.35-3.13 (m, 3H), 3.07 (d, J=18.9 Hz, 1H), 2.68-2.59 (m, 3H), 2.15 (d, J=20.8 Hz, 1H), 1.95-1.74 (m, 2H), 1.69 (d, J=23.3 Hz, 1H), 1.30 (d, J=23.9 Hz, 1H), 1.02-0.93 (m, 3H). ¹³C NMR (151 MHz, CDCl₃) δ 177.25, 156.75, 155.95, 139.33, 138.58, 133.85, 131.84, 128.66, 128.62, 126.71, 126.67, 126.10, 126.06, 125.98, 125.94, 125.59, 125.55, 125.51, 124.05, 124.01, 122.86, 122.42, 50.46, 40.91, 39.13, 30.51, 28.34, 28.30, 22.78, 8.79, 8.74.

HRMS (ESI-TOF) Calcd for C₂₃H₂₆NO₂⁺ [M+H]⁺: 348.1964; found: 348.1962.

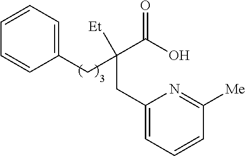

### 2-Ethyl-2-((6-methylpyridin-2-yl)methyl)-5-phenylpentanoic Acid (4u)

Following General Procedure. Purification by pTLC (90% EA/hexanes, Rf=0.35) afforded the title compound as colourless oil (13.7 mg, 0.04 mmol). ¹H NMR (600 MHz, CDCl₃) δ 7.68 (t, J=7.7 Hz, 1H), 7.25 (t, J=7.6 Hz, 2H), 7.20-7.14 (m, 2H), 7.13-7.09 (m, 2H), 7.08 (d, J=7.6 Hz, 1H), 3.04 (d, J=1.6 Hz, 2H), 2.61-2.49 (m, 5H), 1.74-1.60 (m, 4H), 1.53-1.43 (m, 2H), 0.89 (t, J=7.4 Hz, 3H). ¹³C NMR (151 MHz, CDCl₃) δ 177.47, 156.91, 155.82, 142.15, 139.10, 128.38, 128.23, 125.69, 122.66, 122.22, 50.21, 40.68, 36.85, 36.27, 30.35, 26.06, 22.76, 8.75.

HRMS (ESI-TOF) Calcd for C₂₀H₂₆NO₂⁺ [M+H]⁺: 312.1964; found: 312.1974.

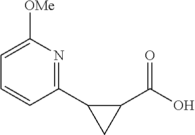 [SMILES: COc1cccc(C2CC2C(=O)O)n1]

### (2R)-2-(6-Methoxypyridin-2-yl)cyclopropane-1-carboxylic Acid (4v)

Following General Procedure. Purification by pTLC (30% EA/hexanes, Rf=0.35) afforded the title compound as colourless oil (8.7 mg, 0.05 mmol). ¹H NMR (600 MHz, CDCl₃) δ 7.63 (t, J=7.9 Hz, 1H), 7.03 (d, J=7.3 Hz, 1H), 6.70 (d, J=8.4 Hz, 1H), 3.95 (s, 3H), 2.46 (q, J=8.4 Hz, 1H), 2.28 (q, J=8.3 Hz, 1H), 1.77 (t, J=8.4 Hz, 2H). ¹³C NMR (151 MHz, CDCl₃) δ 173.66, 163.26, 155.43, 140.56, 117.57, 109.47, 54.20, 25.90, 25.69, 17.18.

HRMS (ESI-TOF) Calcd for C₁₀H₁₂NO₃⁺ [M+H]⁺: 194.0817; found: 194.0820.

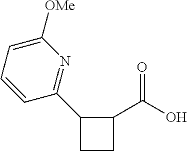 [SMILES: COc1cccc(C2CCC2C(=O)O)n1]

### (2R)-2-(6-Methoxypyridin-2-yl)cyclobutane-1-carboxylic Acid (4w)

Following General Procedure. Purification by pTLC (30% EA/hexanes, Rf=0.35) afforded the title compound as colourless oil (8.5 mg, 0.04 mmol). ¹H NMR (600 MHz, CDCl₃) δ 7.66 (t, J=7.8 Hz, 1H), 6.73 (d, J=6.6 Hz, 2H), 3.95 (s, 3H), 3.77 (q, J=9.3, 8.9 Hz, 1H), 3.27 (td, J=10.1, 7.8 Hz, 1H), 2.47-2.40 (m, 1H), 2.36-2.30 (m, 1H), 2.24-2.09 (m, 2H). ¹³C NMR (151 MHz, CDCl₃) δ 174.48, 163.70, 159.70, 140.43, 112.54, 109.99, 54.28, 44.59, 43.65, 22.35, 21.11.

HRMS (ESI-TOF) Calcd for C₁₁H₁₄NO₃⁺ [M+H]⁺: 208.0974; found: 208.0977.

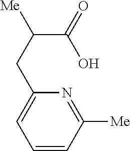 [SMILES: Cc1cccc(CC(C)C(=O)O)n1]

### 3-(6-Methoxypyridin-2-yl)-2-methylpropanoic Acid (4x)

Following General Procedure. Purification by pTLC (40% EA/hexanes, Rf=0.35) afforded the title compound as colourless oil (10.7 mg, 0.06 mmol). ¹H NMR (600 MHz, CDCl₃) δ 7.60 (dd, J=8.4, 7.2 Hz, 1H), 6.81 (d, J=7.2 Hz, 1H), 6.71 (d, J=8.4 Hz, 1H), 3.96 (s, 3H), 3.15 (dd, J=15.4, 8.6 Hz, 1H), 3.05-2.97 (m, 1H), 2.92 (dd, J=15.5, 2.9 Hz, 1H), 1.29 (d, J=7.2 Hz, 3H). ¹³C NMR (151 MHz, CDCl₃) δ 177.57, 163.57, 156.17, 140.16, 116.34, 109.46, 54.18, 39.32, 38.70, 17.40.

HRMS (ESI-TOF) Calcd for C₁₀H₁₄NO₃⁺ [M+H]⁺: 196.0974; found: 196.0981.

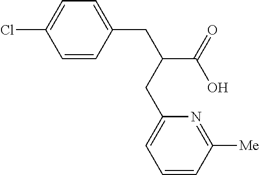 [SMILES: Cc1cccc(CC(Cc2ccc(Cl)cc2)C(=O)O)n1]

### 2-(4-Chlorobenzyl)-3-(6-methylpyridin-2-yl)propanoic Acid (4y)

Following General Procedure. Purification by pTLC (90% EA/hexanes, Rf=0.35) afforded the title compound as colourless oil (14.7 mg, 0.05 mmol). ¹H NMR (600 MHz, CDCl₃) δ 7.64 (t, J=7.7 Hz, 1H), 7.26 (d, J=8.4 Hz, 2H), 7.17 (d, J=7.7 Hz, 1H), 7.09 (d, J=8.2 Hz, 2H), 6.89 (d, J=7.7 Hz, 1H), 3.33 (dd, J=14.3, 5.1 Hz, 1H), 3.12 (td, J=10.3, 5.1 Hz, 1H), 3.03-3.00 (m, 2H), 2.72 (dd, J=14.3, 10.0 Hz, 1H), 2.62 (s, 3H). ¹³C NMR (151 MHz, CDCl₃) δ 175.90, 157.33, 156.38, 139.08, 137.53, 132.29, 130.44, 128.64, 122.40, 121.62, 45.89, 36.53, 35.20, 22.89.

HRMS (ESI-TOF) Calcd for C₁₆H₁₇ClNO₂⁺ [M+H]⁺: 290.0948; found: 290.0941.

Scope of γ-C(sp³)-H heteroarylation of Carboxylic Acids

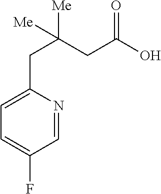 [SMILES: CC(C)(CC(=O)O)Cc1ccc(F)cn1]

### 4-(5-Fluoropyridin-2-yl)-3,3-dimethylbutanoic Acid (6a)

Following General Procedure (reaction concentration is modified to 0.5 M for this substrate). Purification by pTLC (35% EA/hexanes, Rf=0.30) afforded the title compound as colourless oil (10.1 mg, 0.05 mmol). ¹H NMR (600 MHz, CDCl₃) δ 8.43 (d, J=2.9 Hz, 1H), 7.56 (td, J=8.3, 2.9 Hz, 1H), 7.27 (d, J=4.4 Hz, 1H), 2.88 (s, 2H), 2.23 (s, 2H), 1.09 (s, 6H). ¹³C NMR (151 MHz, CDCl₃) δ 172.47, 158.54 (d, J_{CF}=256.7 Hz), 154.32, 135.71 (d, J_{CF}=25.7 Hz), 126.97 (d, J_{CF}=4.5 Hz), 125.35 (d, J_{CF}=18.1 Hz), 46.38, 46.24, 35.21, 28.75. ¹⁹F NMR (376 MHz, CDCl₃) δ −130.24.

HRMS (ESI-TOF) Calcd for C₁₁H₁₅F₁₅NO₂⁺ [M+H]⁺: 212.1087; found: 212.1094.

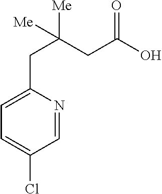 [SMILES: CC(C)(CC(=O)O)Cc1ccc(Cl)cn1]

### 4-(5-Chloropyridin-2-yl)-3,3-dimethylbutanoic Acid (6b)

Following General Procedure (reaction concentration is modified to 0.5 M for this substrate). Purification by pTLC (35% EA/hexanes, Rf=0.30) afforded the title compound as colourless oil (10.0 mg, 0.04 mmol). ¹H NMR (600 MHz, CDCl₃) δ 8.54 (d, J=2.5 Hz, 1H), 7.80 (dd, J=8.4, 2.4 Hz, 1H), 7.24 (d, J=8.3 Hz, 1H), 2.87 (s, 2H), 2.23 (s, 2H), 1.09 (s, 6H). ¹³C NMR (151 MHz, CDCl₃) δ 172.48, 156.40, 146.30, 138.12, 130.92, 126.77, 46.45, 46.37, 35.25, 28.77.

HRMS (ESI-TOF) Calcd for C₁₁H₁₅ClNO₂⁺ [M+H]⁺: 228.0791; found: 228.0797.

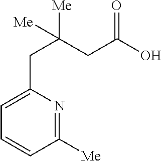 [SMILES: Cc1cccc(CC(C)(C)CC(=O)O)n1]

### 3,3-Dimethyl-4-(6-methylpyridin-2-yl)butanoic Acid (6c)

Following General Procedure (reaction concentration is modified to 0.5 M for this substrate). Purification by pTLC (90% EA/hexanes, Rf=0.30) afforded the title compound as colourless oil (11.6 mg, 0.06 mmol). ¹H NMR (600 MHz, CDCl₃) δ 7.70 (t, J=7.9 Hz, 1H), 7.17 (d, J=7.8 Hz, 1H), 7.07 (d, J=7.3 Hz, 1H), 2.84 (s, 2H), 2.61 (s, 3H), 2.23 (s, 2H), 1.09 (s, 6H). ¹³C NMR (151 MHz, CDCl₃) δ 173.35, 157.05, 156.40, 138.69, 123.03, 122.20, 46.83, 46.66, 35.16, 28.93, 22.91.

HRMS (ESI-TOF) Calcd for C₁₂H₁₈NO₂⁺ [M+H]⁺: 208.1338; found: 208.1347.

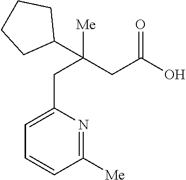 [SMILES: Cc1cccc(CC(C)(CC(=O)O)C2CCCC2)n1]

### 3-Cyclopentyl-3-methyl-4-(6-methylpyridin-2-yl)butanoic Acid (6d)

Following General Procedure (reaction concentration is modified to 0.5 M for this substrate). Purification by pTLC (90% EA/hexanes, Rf=0.30) afforded the title compound as colourless oil (11.5 mg, 0.04 mmol). ¹H NMR (600 MHz, CDCl₃) δ 7.69 (t, J=7.7 Hz, 1H), 7.16 (d, J=7.7 Hz, 1H), 7.09 (d, J=7.6 Hz, 1H), 2.98-2.81 (m, 2H), 2.61 (s, 3H), 2.36-2.22 (m, 2H), 1.75 (tq, J=12.0, 6.4, 4.4 Hz, 3H), 1.61-1.47 (m, 4H), 1.42 (td, J=8.8, 8.2, 2.7 Hz, 1H), 1.33-1.29 (m, 1H), 0.93 (s, 3H). ¹³C NMR (151 MHz, CDCl₃) δ 173.70, 157.16, 156.30, 138.66, 123.08, 122.14, 47.79, 45.17, 45.02, 40.16, 26.88, 25.70, 25.57, 22.86, 20.04.

HRMS (ESI-TOF) Calcd for C₁₆H₂₄NO₂⁺ [M+H]⁺: 262.1802; found: 262.1796.

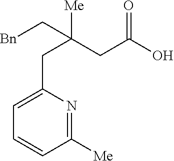 [SMILES: Cc1cccc(CC(C)(CCc2ccccc2)CC(=O)O)n1]

### 3-Methyl-3-((6-methylpyridin-2-yl)methyl)-5-phenylpentanoic acid (6e)

Following General Procedure (reaction concentration is modified to 0.5 M for this substrate). Purification by pTLC (90% EA/hexanes, Rf=0.30) afforded the title compound as colourless oil (12.5 mg, 0.04 mmol). ¹H NMR (600 MHz, CDCl₃) δ 7.70 (t, J=7.7 Hz, 1H), 7.30 (d, J=8.0 Hz, 2H), 7.22-7.16 (m, 4H), 7.09 (d, J=7.6 Hz, 1H), 3.00-2.83 (m, 2H), 2.74 (dtd, J=75.1, 12.9, 5.0 Hz, 2H), 2.62 (s, 3H), 2.41-2.27 (m, 2H), 1.68 (td, J=13.1, 4.8 Hz, 2H), 1.13 (s, 3H). ¹³C NMR (151 MHz, CDCl₃) δ 173.18, 156.65, 156.50, 142.23, 138.74, 128.44, 125.86, 123.17, 122.31, 45.85, 44.55, 43.56, 38.02, 30.15, 25.25, 22.94.

HRMS (ESI-TOF) Calcd for C₁₉H₂₄NO₂⁺ [M+H]⁺: 298.1802; found: 298.1802.

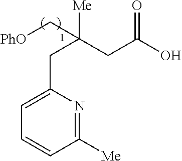

### 3-Methyl-3-((6-methylpyridin-2-yl)methyl)-7-phenoxyheptanoic Acid (6f)

Following General Procedure (reaction concentration is modified to 0.5 M for this substrate). Purification by pTLC (90% EA/hexanes, Rf=0.30) afforded the title compound as colourless oil (17.7 mg, 0.05 mmol). ¹H NMR (600 MHz, CDCl₃) δ 7.61 (t, J=7.7 Hz, 1H), 7.33-7.29 (m, 2H), 7.15 (d, J=7.8 Hz, 1H), 7.06 (d, J=7.6 Hz, 1H), 6.96 (t, J=7.3 Hz, 1H), 6.92 (d, J=7.9 Hz, 2H), 3.99 (t, J=6.3 Hz, 2H), 2.95-2.78 (m, 2H), 2.32-2.20 (m, 2H), 1.85-1.73 (m, 2H), 1.64-1.59 (m, 2H), 1.39 (q, J=7.4 Hz, 2H), 1.04 (s, 3H). ¹³C NMR (151 MHz, CDCl₃) δ 173.46, 159.01, 156.77, 156.33, 138.79, 129.46, 123.14, 122.26, 120.59, 114.49, 67.35, 45.19, 40.65, 37.92, 29.71, 25.16, 22.86, 20.11.

HRMS (ESI-TOF) Calcd for C₂₁H₂₈NO₃⁺ [M+H]⁺: 342.2064; found: 342.2058.

### Representative Synthetic Applications

### Di-Functionalization of Isobutyric Acid

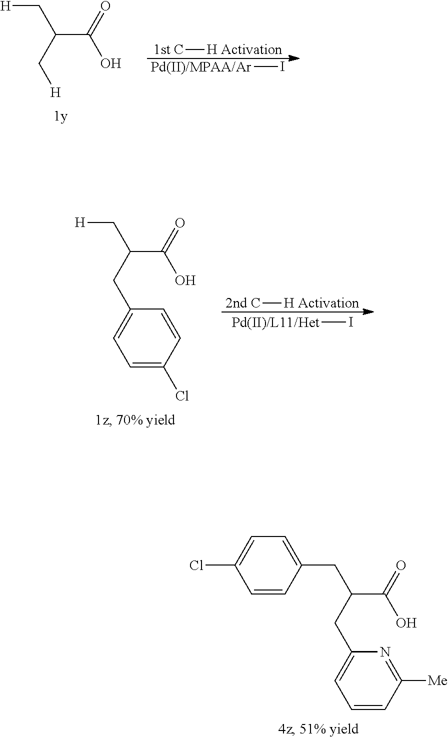

### Product 1z was Synthesized Following the Reported Procedure in Reference:⁸

Carboxylic acid (1y, 0.2 mmol), 1-Chloro-4-iodobenzene (0.1 mmol), Pd(OAc)₂ (10 mol %), Ac-Gly-OH (30 mol %), Ag₂CO₃ (1.0 equiv.), K₂CO₃ (0.5 equiv.) and HFIP (1.0 ml) were added to a reaction vial (10 ml). The vial was capped under air and closed tightly. Then the reaction mixture was stirred at 80° C. for 12 hours. After cooling to room temperature, the mixture was acidified with AcOH (5.0 equiv.) and filtered through a pad of celite with acetone as the eluent to remove the insoluble precipitate. The resulting solution was concentrated and purified by preparative thin-layer chromatography to afford the desired product.

### Product 4y was Synthesized Following the General Procedure,

### Diverse Functionalization of Pivalic Acid

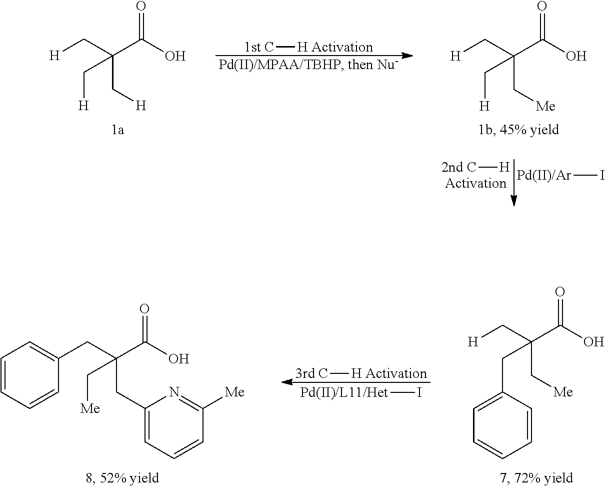

### Product 1b was Synthesized Following the Reported Procedure in Reference: 5

In the culture tube, Pd(CH₃CN)₂Cl₂ (10 mol %), MPAA Ligand (20 mol %), CsHCO₃ (0.5 eq), and carboxylic acid 1a (0.1 mmol) in order were weighed in air and placed with a magnetic stir bar. Then HFIP (1.0 mL) and TBHP (ca. 5.5 M in decane) (2.0 eq, 36 μL) were added. The reaction mixture was stirred at rt for 3 min, and then heated to 60° C. for 12 h (600 rpm). After being allowed to cool to room temperature, the mixture was filtered through a pad of celite with acetone as the eluent to remove the insoluble precipitate and concentrated in vacuo, the crude product was used directly in next step without further purification.

In the culture tube, CuBr₂·Me₂S (20 mol %, 4.1 mg), B-lactone (0.1 mmol, 24.8 mg), and Me₂S (1.0 eq, 7 μL) in order were added and placed with a magnetic stir bar. Then THF (1.0 mL) was added. The reaction mixture was stirred at rt for 3 min, and then Grignard reagent (3.0 eq) was added dropwise at 0° C. After being allowed to stir at 0° C. in 1 h, the mixture was quenched with saturated NH₄Cl solution. The resulting mixture was diluted with EA, washed with saturated NH₄Cl solution, and dried with MgSO₄. After being concentrated in vacuo, the resulting mixture was purified by pTLC.

### Product 7 was Synthesized Following the Reported Procedure in Reference: 9

Carboxylic acid 1b (0.2 mmol), aryl iodide (0.4 mmol), Ag₂CO₃ (0.4 mmol), K₂HPO₄ (0.2 mmol) and sodium acetate (0.5 mmol) were dissolved in tert-butanol (2.5 mL) in a 10 mL vessel. Pd(OAc)₂ (0.02 mmol) was added to the reaction mixture, tightly capped and heated to 120° C. with vigorous stirring. The reaction was stopped after it completely turned black. The black solid was filtered off, the filtrate was basified with 0.5 N NaOH and tert-butanol was removed in a rotary evaporator. The aqueous fraction was washed with diethyl ether (2 mL×3), acidified with 2N HCl and extracted with ethyl acetate (5 mL×5). The combined ethyl acetate fraction was dried over Na₂SO₄ and the solvent was removed in a rotary evaporator. The crude mixture was roughly purified by pTLC and used in the next step.

Product 8 was Synthesized Following the General Procedure. See Section 3.

¹H NMR (600 MHz, CDCl₃) δ 7.68 (t, J=7.7 Hz, 1H), 7.28-7.17 (m, 5H), 7.14 (d, J=7.8 Hz, 1H), 7.09 (d, J=7.7 Hz, 1H), 3.32 (d, J=13.6 Hz, 1H), 3.01 (d, J=15.6 Hz, 1H), 2.93 (d, J=15.6 Hz, 1H), 2.71 (d, J=13.6 Hz, 1H), 2.56 (s, 3H), 1.68 (dq, J=14.9, 7.5 Hz, 1H), 1.50 (dq, J=14.6, 7.4 Hz, 1H), 0.95 (t, J=7.4 Hz, 3H). ¹³C NMR (151 MHz, CDCl₃) δ 176.88, 156.46, 155.17, 138.76, 137.17, 130.04, 127.62, 125.99, 122.50, 121.84, 51.41, 43.33, 40.54, 30.62, 22.12, 8.48.

HRMS (ESI-TOF) Calcd for C₁₈H₂₂NO₂⁺ [M+H]⁺: 284.1645; found: 284.1645.

The foregoing disclosure has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the disclosure should, therefore, be determined not with reference to the above description, but should instead be determined with reference to the following appended claims, along with the full scope of equivalents to which such claims are entitled.

This application refers to various issued patents, published patent applications, journal articles, and other publications, each of which are incorporated herein by reference.

What is claimed is:

1. A method of β- or γ-C(sp³)-H heteroarylation of aliphatic carboxylic acids, comprising treating an aliphatic carboxylic acid with a bidentate pyridine-pyridone ligand in the presence of a Pd(II) catalyst.

2. The method of claim 1, wherein the method of β- or γ-C(sp³)-H heteroarylation of aliphatic carboxylic acids occurs according to the following reaction scheme: 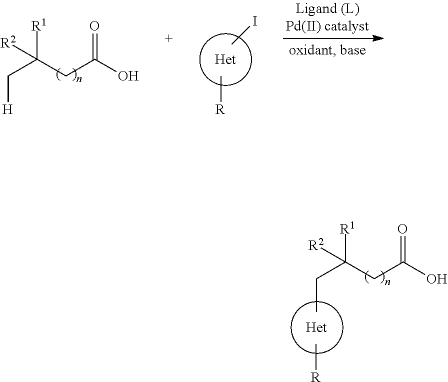
wherein:
Het is (C₅-C₆)heteroaryl;
R is H, halo, OH, (C₁-C₆)alkyl, halo (C₁-C₆)alkyl, halo (C₁-C₆)heteroalkyl, Ac, or —C(═O)H;
R¹ and R² are independently H or selected from the group consisting of (C₁-C₆)alkyl, (C₁-C₆)heteroalkyl, (C₃-C₇)cycloalkyl, (C₃-C₇)heterocycloalkyl, (C₆-C₁₀)aryl, (C₅-C₆)heteroaryl, (C₁-C₆)alkyl (C₃-C₇)cycloalkyl, (C₁-C₆)alkyl (C₃-C₇)heterocycloalkyl, (C₁-C₆)alkyl (C₆-C₁₀)aryl, (C₁-C₆)alkyl (C₅-C₆)heteroaryl, halo (C₁-C₆)alkyl, halo (C₁-C₆)heteroalkyl, halo (C₃-C₇)cycloalkyl, halo (C₃-C₇)heterocycloalkyl, halo (C₆-C₁₀)aryl, halo (C₅-C₆)heteroaryl, halo (C₁-C₆)alkyl (C₃-C₇)cycloalkyl, halo (C₁-C₆)alkyl (C₃-C₇) heterocycloalkyl, halo (C₁-C₆)alkyl (C₆-C₁₀)aryl, and halo (C₁-C₆)alkyl (C₅-C₆)heteroaryl, each independently and optionally substituted with one or more R′;
each R′ is independently OH, halo, CN, (C₁-C₆)alkyl, halo (C₁-C₆)alkyl, (C₁-C₆)heteroalkyl, (C₃-C₇)cycloalkyl, (C₃-C₇)heterocycloalkyl, NH₂, or —S(═O)₂Me; and
n is 0 or 1.

3. The method of claim 2, wherein n is 0.

4. The method of claim 2, wherein n is 1.

5. The method of any one of claims 2-4, wherein Het is pyridinyl.

6. The method of any one of claims 2-4, wherein Het is pyrazinyl.

7. The method of any one of claims 2-4, wherein Het is furanyl.

8. The method of any one of claims 2-4, wherein Het is thiophenyl.

9. The method of any one of claims 2-8, wherein R is H.

10. The method of any one of claims 2-8, wherein R is Me.

11. The method of any one of claims 2-8, wherein R is Cl.

12. The method of any one of claims 2-8, wherein R is F.

13. The method of any one of claims 2-8, wherein R is Br.

14. The method of any one of claims 2-8, wherein R is CF₃.

15. The method of any one of claims 2-8, wherein R is OMe.

16. The method of any one of claims 2-8, wherein R is —C(═O)H or Ac.

17. The method of any one of claims 2-16, wherein R¹ is H.

18. The method of any one of claims 2-16, wherein R¹ is (C₁-C₆)alkyl.

19. The method of claim 18, wherein R¹ is Me.

20. The method of claim 18, wherein R¹ is Et.

21. The method of claim 18, wherein R¹ is Pr.

22. The method of claim 18, wherein R¹ is iPr.

23. The method of claim 18, wherein R¹ is Bu.

24. The method of any one of claims 2-16, wherein R¹ is (C₁-C₆)heteroalkyl.

25. The method of any one of claims 2-16, wherein R¹ is (C₆-C₁₀)aryl optionally substituted with one or more R′.

26. The method of claim 25, wherein R¹ is Ph optionally substituted with one or more R′.

27. The method of any one of claims 2-16, wherein R¹ is (C₁-C₆)alkyl (C₆-C₁₀)aryl optionally substituted with one or more R′.

28. The method of claim 27, wherein R¹ is Bn optionally substituted with one or more R′.

29. The method of any one of claims 25-18, wherein R′ is Me or halo.

30. The method of any one of claims 2-29, wherein R² is H.

31. The method of any one of claims 2-29, wherein R² is (C₁-C₆)alkyl.

32. The method of claim 31, wherein R² is Me.

33. The method of claim 31, wherein R² is Et.

34. The method of claim 31, wherein R² is Pr.

35. The method of claim 31, wherein R² is iPr.

36. The method of claim 31, wherein R² is Bu.

37. The method of any one of claims 2-29, wherein R² is (C₁-C₆)heteroalkyl.

38. The method of any one of claims 2-29, wherein R² is (C₆-C₁₀)aryl optionally substituted with one or more R′.

39. method of claim 38, wherein R² is Ph optionally substituted with one or more R′.

40. The method of any one of claims 2-29, wherein R² is (C₁-C₆)alkyl (C₆-C₁₀)aryl optionally substituted with one or more R′.

41. The method of claim 40, wherein R² is Bn optionally substituted with one or more R′.

42. The method of any one of claims 2-29, wherein R² is halo (C₁-C₆)alkyl (C₆-C₁₀)aryl.

43. The method of claim 2, wherein R² and R³ together form (C₃-C₇)cycloalkyl.

44. The method of claim 2, wherein R² and R³ together form cyclopropyl.

45. The method of claim 2, wherein R² and R³ together form cyclobutyl.

46. The method of claim 6, wherein R² is (C₃-C₆)cycloalkyl.

47. The method of any one of claims 2-46, wherein L is selected from the group consisting of:

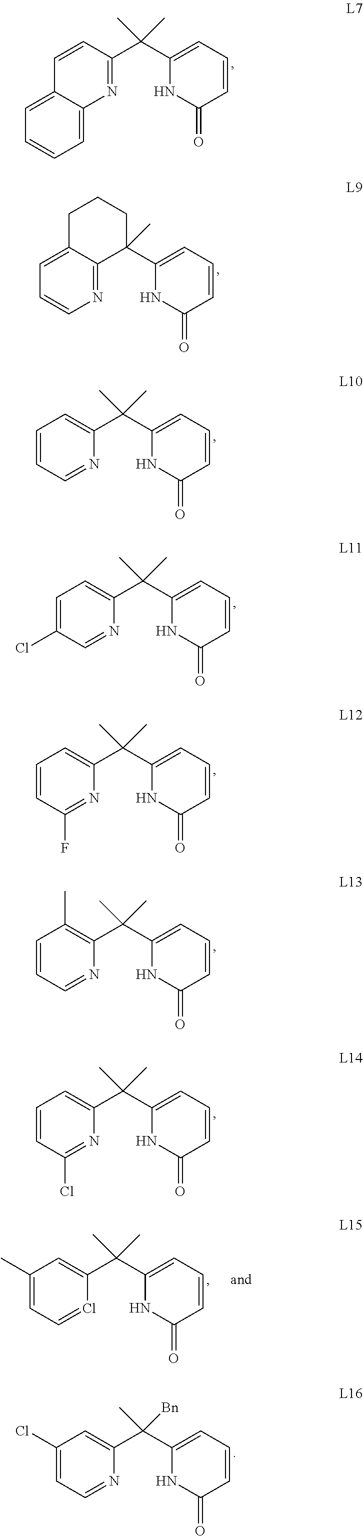

48. The method of claim 47, wherein L is L11.

49. The method of claim 2, wherein the method of β- or γ-C(sp³)-H heteroarylation of aliphatic carboxylic acids occurs according to the following reaction scheme: 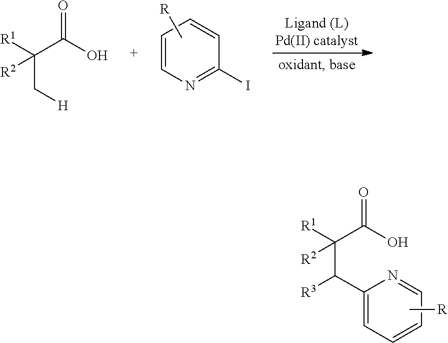
wherein:
R³ is H;
or R² and R³ together form (C₃-C₄)cycloalkyl.

50. The method of claim 2, wherein the method of β- or γ-C(sp³)-H heteroarylation of aliphatic carboxylic acids occurs according to the following reaction scheme:

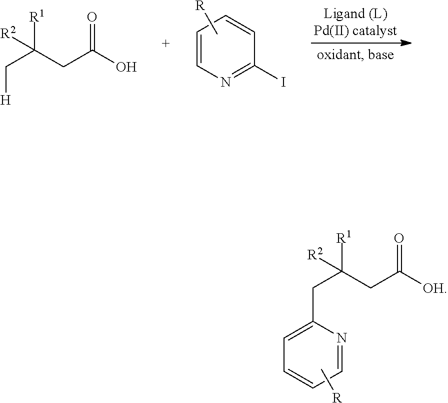

51. The method of either claim 49 or claim 50, wherein Lis L11.

52. The method of any one of claims 49-51, wherein R¹ is (C₁-C₆)alkyl.

53. The method of any one of claims 49-52, wherein R² is (C₁-C₆)alkyl.

54. The method of any one of claims 49-53, wherein R is H.

55. The method of any one of claims 49-53, wherein R is Me.

56. The method of any one of claims 49-53, wherein R is Cl.

57. The method of any one of claims 49-53, wherein R is F.

58. The method of any one of claims 49-53, wherein R is Br.

59. The method of any one of claims 49-53, wherein R is CF₃.

60. The method of any one of claims 49-53, wherein R is OMe.

61. The method any one of claims 2-60, wherein the Pd(II) catalyst is Pd(OAc)₂.

62. The method any one of claims 2-61, wherein the oxidant is Ag₂CO₃.

63. The method any one of claims 2-62, wherein the base is KH₂PO₄.

64. The method any one of claims 2-63, wherein the solvent is HFIP.

65. The method any one of claims 2-64, wherein the concentration is approximately 1.0 M.

66. The method of claim 2, wherein the method of β- or γ-C(sp³)-H heteroarylation of aliphatic carboxylic acids occurs according to the following reaction scheme:

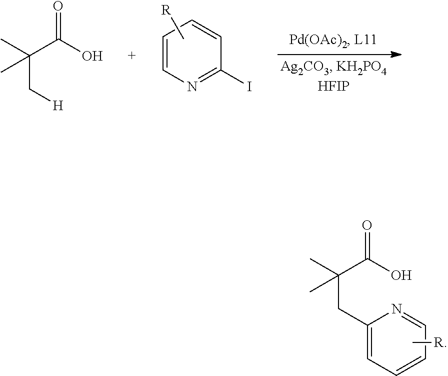

67. The method of claim 2, wherein the method of β- or γ-C(sp³)-H heteroarylation of aliphatic carboxylic acids occurs according to the following reaction scheme:

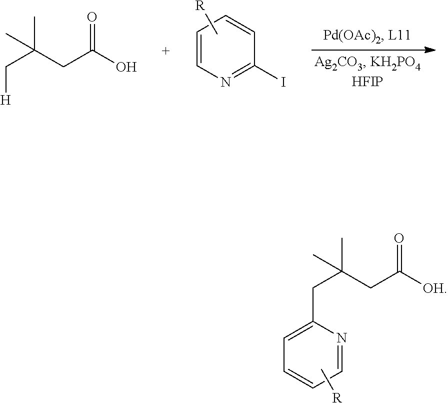

68. The method of any one of claims 2-67, wherein the reaction temperature is between approximately 110-140° C.

69. method of claim 68, wherein the reaction temperature is approximately 120° C.

70. Any method of β- or γ-C(sp³)-H heteroarylation of aliphatic carboxylic acids as disclosed herein.
